SEQUENCE LISTING (304 sequences)

SEQ ID NO: 1 - DNA, 21 bp, Artificial Sequence
  Oligonulceotide
ucauugagtgtcctcugccuc

SEQ ID NO: 2 - DNA, 21 bp, Artificial Sequence
  Oligonulceotide
uagguacacgtaggacuugcc

SEQ ID NO: 3 - DNA, 21 bp, Artificial Sequence
  Oligonulceotide
aauauuaggatggtgucuguc

SEQ ID NO: 4 - DNA, 21 bp, Artificial Sequence
  Oligonulceotide
gaaguugccgggagagcugaa

SEQ ID NO: 5 - DNA, 21 bp, Artificial Sequence
  Oligonulceotide
gguugctgtctttccugggug

SEQ ID NO: 6 - DNA, 21 bp, Artificial Sequence
  Oligonulceotide
aguugctgctttagguggagu

SEQ ID NO: 7 - DNA, 21 bp, Artificial Sequence
  Oligonulceotide
gaugugactgtcttccaaguc

SEQ ID NO: 8 - DNA, 21 bp, Artificial Sequence
  Oligonulceotide
ugaauucagtactgcagagcu

SEQ ID NO: 9 - DNA, 21 bp, Artificial Sequence
  Oligonulceotide
cuuugactggaaagtucaucu

SEQ ID NO: 10 - DNA, 21 bp, Artificial Sequence
  Oligonulceotide
ugacccuatgctgtuccuaua

SEQ ID NO: 11 - DNA, 21 bp, Artificial Sequence
  Oligonulceotide
tcattgagtgtcctctgcctc

SEQ ID NO: 12 - DNA, 21 bp, Artificial Sequence
  Oligonulceotide
taggtacacgtaggacttgcc

SEQ ID NO: 13 - DNA, 21 bp, Artificial Sequence
  Oligonulceotide
aatattaggatggtgtctgtc

SEQ ID NO: 14 - DNA, 21 bp, Artificial Sequence
  Oligonulceotide
gaagttgccgggagagctgaa

SEQ ID NO: 15 - DNA, 21 bp, Artificial Sequence
  Oligonulceotide
ggttgctgtctttcctgggtg

SEQ ID NO: 16 - DNA, 21 bp, Artificial Sequence
  Oligonulceotide
agttgctgctttaggtggagt

SEQ ID NO: 17 - DNA, 21 bp, Artificial Sequence
  Oligonulceotide
gatgtgactgtcttccaagtc

SEQ ID NO: 18 - DNA, 21 bp, Artificial Sequence
  Oligonulceotide
tgaattcagtactgcagagct

SEQ ID NO: 19 - DNA, 21 bp, Artificial Sequence
  Oligonulceotide
ctttgactggaaagttcatct

SEQ ID NO: 20 - DNA, 21 bp, Artificial Sequence
  Oligonulceotide
tgaccctatgctgttcctata

SEQ ID NO: 21 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ucauugagtgtcctcugccuc

SEQ ID NO: 22 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uagguacacgtaggacuugcc

SEQ ID NO: 23 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aauauuaggatggtgucuguc

SEQ ID NO: 24 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
gaaguugccgggagagcugaa

SEQ ID NO: 25 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
gguugctgtctttccugggug

SEQ ID NO: 26 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aguugctgctttagguggagu

SEQ ID NO: 27 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
gaugugactgtcttccaaguc

SEQ ID NO: 28 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ugaauucagtactgcagagcu

SEQ ID NO: 29 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
cuuugactggaaagtucaucu

SEQ ID NO: 30 - DNA, 21 bp, Artificial Sequence
  Oligonulceotide
  misc_feature: Residues 1-7, 15-20 are FANA modified bases with Phosphorothioate Linkage; Residues 8-14 are bases with Phosphorothioate Linkage; Residue 21 is FANA modified base
ugacccuatgctgtuccuaua

SEQ ID NO: 31 - DNA, 21 bp, Artificial Sequence
  Oligonulceotide
agaugaagccttggtcagugc

SEQ ID NO: 32 - DNA, 21 bp, Artificial Sequence
  Oligonulceotide
uuucutgcggaactccagcuc

SEQ ID NO: 33 - DNA, 21 bp, Artificial Sequence
  Oligonulceotide
acaggtgaattctaacaggcc

SEQ ID NO: 34 - DNA, 21 bp, Artificial Sequence
  Oligonulceotide
uaguucctctgcagtcuaagc

SEQ ID NO: 35 - DNA, 21 bp, Artificial Sequence
  Oligonulceotide
auaaatgagtagttccucugc

SEQ ID NO: 36 - DNA, 21 bp, Artificial Sequence
  Oligonulceotide
uuugagtgtactgaggcaggc

SEQ ID NO: 37 - DNA, 21 bp, Artificial Sequence
  Oligonulceotide
guucaaggaagaagaggaggc

SEQ ID NO: 38 - DNA, 21 bp, Artificial Sequence
  Oligonulceotide
acugaccaaggcttcaucugu

SEQ ID NO: 39 - DNA, 21 bp, Artificial Sequence
  Oligonulceotide
aaacagcacattcccagaguu

SEQ ID NO: 40 - DNA, 21 bp, Artificial Sequence
  Oligonulceotide
uugugaaggctctgttuggcu

SEQ ID NO: 41 - DNA, 21 bp, Artificial Sequence
  Oligonulceotide
guugugaaggctctgtuuggc

SEQ ID NO: 42 - DNA, 21 bp, Artificial Sequence
  Oligonulceotide
guuguttgagtgtactgaggc

SEQ ID NO: 43 - DNA, 21 bp, Artificial Sequence
  Oligonulceotide
aggugaattctaacaggccgu

SEQ ID NO: 44 - DNA, 21 bp, Artificial Sequence
  Oligonulceotide
gucuaagctgaggcatggauc

SEQ ID NO: 45 - DNA, 21 bp, Artificial Sequence
  Oligonulceotide
agucuaagctgaggcauggau

SEQ ID NO: 46 - DNA, 21 bp, Artificial Sequence
  Oligonulceotide
gaugatgccacagatgaagcc

SEQ ID NO: 47 - DNA, 21 bp, Artificial Sequence
  Oligonulceotide
uguugagtgagggacaggauu

SEQ ID NO: 48 - DNA, 21 bp, Artificial Sequence
  Oligonulceotide
aaccucaaagctgcatcauca

SEQ ID NO: 49 - DNA, 21 bp, Artificial Sequence
  Oligonulceotide
cacaggtgaattctaacaggc

SEQ ID NO: 50 - DNA, 21 bp, Artificial Sequence
  Oligonulceotide
agucuaagctgaggcauggau

SEQ ID NO: 51 - DNA, 21 bp, Artificial Sequence
  Oligonulceotide
auguaccaaggcaggcucuuc

SEQ ID NO: 52 - DNA, 21 bp, Artificial Sequence
  Oligonulceotide
aauautaggatggtgtcuguc

SEQ ID NO: 53 - DNA, 21 bp, Artificial Sequence
  Oligonulceotide
uauagctggttgtgagggcuc

SEQ ID NO: 54 - DNA, 21 bp, Artificial Sequence
  Oligonulceotide
ucauugagtgtcctctgccuc

SEQ ID NO: 55 - DNA, 21 bp, Artificial Sequence
  Oligonulceotide
acuaacagctagaggcuuugc

SEQ ID NO: 56 - DNA, 21 bp, Artificial Sequence
  Oligonulceotide
ccacutgcagactccauuugc

SEQ ID NO: 57 - DNA, 21 bp, Artificial Sequence
  Oligonulceotide
auaggtacacgtaggacuugc

SEQ ID NO: 58 - DNA, 21 bp, Artificial Sequence
  Oligonulceotide
auuucattgagtgtccucugc

SEQ ID NO: 59 - DNA, 21 bp, Artificial Sequence
  Oligonulceotide
uagautgagacagagaugggc

SEQ ID NO: 60 - DNA, 21 bp, Artificial Sequence
  Oligonulceotide
ugacuggatgtaagtauaggc

SEQ ID NO: 61 - DNA, 21 bp, Artificial Sequence
  Oligonulceotide
uguucaaggtatggagcaggc

SEQ ID NO: 62 - DNA, 21 bp, Artificial Sequence
  Oligonulceotide
aguucacgaatgtaccaaggc

SEQ ID NO: 63 - DNA, 21 bp, Artificial Sequence
  Oligonulceotide
uguuacgggaataggaggagc

SEQ ID NO: 64 - DNA, 21 bp, Artificial Sequence
  Oligonulceotide
cuaacagctagaggctuugcc

SEQ ID NO: 65 - DNA, 21 bp, Artificial Sequence
  Oligonulceotide
uagguacacgtaggacuugcc

SEQ ID NO: 66 - DNA, 21 bp, Artificial Sequence
  Oligonulceotide
uuucattgagtgtcctcugcc

SEQ ID NO: 67 - DNA, 21 bp, Artificial Sequence
  Oligonulceotide
auagutaccctggtttcuacc

SEQ ID NO: 68 - DNA, 21 bp, Artificial Sequence
  Oligonulceotide
uucuaccagatcttgtcggac

SEQ ID NO: 69 - DNA, 21 bp, Artificial Sequence
  Oligonulceotide
uucuagcactctctttcauuu

SEQ ID NO: 70 - DNA, 21 bp, Artificial Sequence
  Oligonulceotide
aaguataggcacgtgaguguu

SEQ ID NO: 71 - DNA, 21 bp, Artificial Sequence
  Oligonulceotide
ucacuactaggcagagcuguu

SEQ ID NO: 72 - DNA, 21 bp, Artificial Sequence
  Oligonulceotide
acaactaacagctagaggcuu

SEQ ID NO: 73 - DNA, 21 bp, Artificial Sequence
  Oligonulceotide
acacucacgtgcctatacuua

SEQ ID NO: 74 - DNA, 21 bp, Artificial Sequence
  Oligonulceotide
uucauttggtatccgcuuucu

SEQ ID NO: 75 - DNA, 21 bp, Artificial Sequence
  Oligonulceotide
agugagacgtggggtacaucu

SEQ ID NO: 76 - DNA, 21 bp, Artificial Sequence
  Oligonulceotide
ugguatccgctttctccugcu

SEQ ID NO: 77 - DNA, 21 bp, Artificial Sequence
  Oligonulceotide
uucautgagtgtcctcugccu

SEQ ID NO: 78 - DNA, 21 bp, Artificial Sequence
  Oligonulceotide
acuagtaaagggtggtcuuau

SEQ ID NO: 79 - DNA, 21 bp, Artificial Sequence
  Oligonulceotide
auagugcccgtggttccagau

SEQ ID NO: 80 - DNA, 21 bp, Artificial Sequence
  Oligonulceotide
uguuctagcactctctuucau

SEQ ID NO: 81 - DNA, 21 bp, Artificial Sequence
  Oligonulceotide
uauuaggatggtgtctgucau

SEQ ID NO: 82 - DNA, 21 bp, Artificial Sequence
  Oligonulceotide
caauacctctctgccacuuuc

SEQ ID NO: 83 - DNA, 21 bp, Artificial Sequence
  Oligonulceotide
cuaagaaggatgatgcuguuc

SEQ ID NO: 84 - DNA, 21 bp, Artificial Sequence
  Oligonulceotide
cacuactaggcagagcuguuc

SEQ ID NO: 85 - DNA, 21 bp, Artificial Sequence
  Oligonulceotide
agaugaagccttggtcagugc

SEQ ID NO: 86 - DNA, 21 bp, Artificial Sequence
  Oligonulceotide
uuucuugcggaactccagcuc

SEQ ID NO: 87 - DNA, 21 bp, Artificial Sequence
  Oligonulceotide
acaggugaattctaacaggcc

SEQ ID NO: 88 - DNA, 21 bp, Artificial Sequence
  Oligonulceotide
uaguucctctgcagtcuaagc

SEQ ID NO: 89 - DNA, 21 bp, Artificial Sequence
  Oligonulceotide
auaaaugagtagttccucugc

SEQ ID NO: 90 - DNA, 21 bp, Artificial Sequence
  Oligonulceotide
uuugagtgtactgaggcaggc

SEQ ID NO: 91 - DNA, 21 bp, Artificial Sequence
  Oligonulceotide
guucaaggaagaagaggaggc

SEQ ID NO: 92 - DNA, 21 bp, Artificial Sequence
  Oligonulceotide
acugaccaaggcttcaucugu

SEQ ID NO: 93 - DNA, 21 bp, Artificial Sequence
  Oligonulceotide
aaacagcacattcccagaguu

SEQ ID NO: 94 - DNA, 21 bp, Artificial Sequence
  Oligonulceotide
uugugaaggctctgtuuggcu

SEQ ID NO: 95 - DNA, 21 bp, Artificial Sequence
  Oligonulceotide
guugugaaggctctguuuggc

SEQ ID NO: 96 - DNA, 21 bp, Artificial Sequence
  Oligonulceotide
guuguutgagtgtacugaggc

SEQ ID NO: 97 - DNA, 21 bp, Artificial Sequence
  Oligonulceotide
aggugaattctaacaggccgu

SEQ ID NO: 98 - DNA, 21 bp, Artificial Sequence
  Oligonulceotide
gucuaagctgaggcauggauc

SEQ ID NO: 99 - DNA, 21 bp, Artificial Sequence
  Oligonulceotide
agucuaagctgaggcauggau

SEQ ID NO: 100 - DNA, 21 bp, Artificial Sequence
  Oligonulceotide
gaugaugccacagatgaagcc

SEQ ID NO: 101 - DNA, 21 bp, Artificial Sequence
  Oligonulceotide
uguugagtgagggacaggauu

SEQ ID NO: 102 - DNA, 21 bp, Artificial Sequence
  Oligonulceotide
aaccucaaagctgcatcauca

SEQ ID NO: 103 - DNA, 21 bp, Artificial Sequence
  Oligonulceotide
cacaggtgaattctaacaggc

SEQ ID NO: 104 - DNA, 21 bp, Artificial Sequence
  Oligonulceotide
agucuaagctgaggcauggau

SEQ ID NO: 105 - DNA, 21 bp, Artificial Sequence
  Oligonulceotide
auguaccaaggcaggcucuuc

SEQ ID NO: 106 - DNA, 21 bp, Artificial Sequence
  Oligonulceotide
aauauuaggatggtgucuguc

SEQ ID NO: 107 - DNA, 21 bp, Artificial Sequence
  Oligonulceotide
uauagctggttgtgagggcuc

SEQ ID NO: 108 - DNA, 21 bp, Artificial Sequence
  Oligonulceotide
ucauugagtgtcctcugccuc

SEQ ID NO: 109 - DNA, 21 bp, Artificial Sequence
  Oligonulceotide
acuaacagctagaggcuuugc

SEQ ID NO: 110 - DNA, 21 bp, Artificial Sequence
  Oligonulceotide
ccacuugcagactccauuugc

SEQ ID NO: 111 - DNA, 21 bp, Artificial Sequence
  Oligonulceotide
auagguacacgtaggacuugc

SEQ ID NO: 112 - DNA, 21 bp, Artificial Sequence
  Oligonulceotide
auuucattgagtgtccucugc

SEQ ID NO: 113 - DNA, 21 bp, Artificial Sequence
  Oligonulceotide
uagauugagacagagaugggc

SEQ ID NO: 114 - DNA, 21 bp, Artificial Sequence
  Oligonulceotide
ugacuggatgtaagtauaggc

SEQ ID NO: 115 - DNA, 21 bp, Artificial Sequence
  Oligonulceotide
uguucaaggtatggagcaggc

SEQ ID NO: 116 - DNA, 21 bp, Artificial Sequence
  Oligonulceotide
aguucacgaatgtaccaaggc

SEQ ID NO: 117 - DNA, 21 bp, Artificial Sequence
  Oligonulceotide
uguuacgggaataggaggagc

SEQ ID NO: 118 - DNA, 21 bp, Artificial Sequence
  Oligonulceotide
cuaacagctagaggcuuugcc

SEQ ID NO: 119 - DNA, 21 bp, Artificial Sequence
  Oligonulceotide
uagguacacgtaggacuugcc

SEQ ID NO: 120 - DNA, 21 bp, Artificial Sequence
  Oligonulceotide
uuucautgagtgtccucugcc

SEQ ID NO: 121 - DNA, 21 bp, Artificial Sequence
  Oligonulceotide
auaguuaccctggttucuacc

SEQ ID NO: 122 - DNA, 21 bp, Artificial Sequence
  Oligonulceotide
uucuaccagatcttgucggac

SEQ ID NO: 123 - DNA, 21 bp, Artificial Sequence
  Oligonulceotide
uucuagcactctcttucauuu

SEQ ID NO: 124 - DNA, 21 bp, Artificial Sequence
  Oligonulceotide
aaguauaggcacgtgaguguu

SEQ ID NO: 125 - DNA, 21 bp, Artificial Sequence
  Oligonulceotide
ucacuactaggcagagcuguu

SEQ ID NO: 126 - DNA, 21 bp, Artificial Sequence
  Oligonulceotide
acaacuaacagctagaggcuu

SEQ ID NO: 127 - DNA, 21 bp, Artificial Sequence
  Oligonulceotide
acacucacgtgcctauacuua

SEQ ID NO: 128 - DNA, 21 bp, Artificial Sequence
  Oligonulceotide
uucauutggtatccgcuuucu

SEQ ID NO: 129 - DNA, 21 bp, Artificial Sequence
  Oligonulceotide
agugagacgtggggtacaucu

SEQ ID NO: 130 - DNA, 21 bp, Artificial Sequence
  Oligonulceotide
ugguauccgctttctccugcu

SEQ ID NO: 131 - DNA, 21 bp, Artificial Sequence
  Oligonulceotide
uucauugagtgtcctcugccu

SEQ ID NO: 132 - DNA, 21 bp, Artificial Sequence
  Oligonulceotide
acuaguaaagggtggucuuau

SEQ ID NO: 133 - DNA, 21 bp, Artificial Sequence
  Oligonulceotide
auagugcccgtggttccagau

SEQ ID NO: 134 - DNA, 21 bp, Artificial Sequence
  Oligonulceotide
uguucuagcactctcuuucau

SEQ ID NO: 135 - DNA, 21 bp, Artificial Sequence
  Oligonulceotide
uauuaggatggtgtcugucau

SEQ ID NO: 136 - DNA, 21 bp, Artificial Sequence
  Oligonulceotide
caauacctctctgccacuuuc

SEQ ID NO: 137 - DNA, 21 bp, Artificial Sequence
  Oligonulceotide
cuaagaaggatgatgcuguuc

SEQ ID NO: 138 - DNA, 21 bp, Artificial Sequence
  Oligonulceotide
cacuactaggcagagcuguuc

SEQ ID NO: 139 - DNA, 21 bp, Artificial Sequence
  Oligonulceotide
agatgaagccttggtcagtgc

SEQ ID NO: 140 - DNA, 21 bp, Artificial Sequence
  Oligonulceotide
tttcttgcggaactccagctc

SEQ ID NO: 141 - DNA, 21 bp, Artificial Sequence
  Oligonulceotide
acaggtgaattctaacaggcc

SEQ ID NO: 142 - DNA, 21 bp, Artificial Sequence
  Oligonulceotide
tagttcctctgcagtctaagc

SEQ ID NO: 143 - DNA, 21 bp, Artificial Sequence
  Oligonulceotide
ataaatgagtagttcctctgc

SEQ ID NO: 144 - DNA, 21 bp, Artificial Sequence
  Oligonulceotide
tttgagtgtactgaggcaggc

SEQ ID NO: 145 - DNA, 21 bp, Artificial Sequence
  Oligonulceotide
gttcaaggaagaagaggaggc

SEQ ID NO: 146 - DNA, 21 bp, Artificial Sequence
  Oligonulceotide
actgaccaaggcttcatctgt

SEQ ID NO: 147 - DNA, 21 bp, Artificial Sequence
  Oligonulceotide
aaacagcacattcccagagtt

SEQ ID NO: 148 - DNA, 21 bp, Artificial Sequence
  Oligonulceotide
ttgtgaaggctctgtttggct

SEQ ID NO: 149 - DNA, 21 bp, Artificial Sequence
  Oligonulceotide
gttgtgaaggctctgtttggc

SEQ ID NO: 150 - DNA, 21 bp, Artificial Sequence
  Oligonulceotide
gttgtttgagtgtactgaggc

SEQ ID NO: 151 - DNA, 21 bp, Artificial Sequence
  Oligonulceotide
aggtgaattctaacaggccgt

SEQ ID NO: 152 - DNA, 21 bp, Artificial Sequence
  Oligonulceotide
gtctaagctgaggcatggatc

SEQ ID NO: 153 - DNA, 21 bp, Artificial Sequence
  Oligonulceotide
agtctaagctgaggcatggat

SEQ ID NO: 154 - DNA, 21 bp, Artificial Sequence
  Oligonulceotide
gatgatgccacagatgaagcc

SEQ ID NO: 155 - DNA, 21 bp, Artificial Sequence
  Oligonulceotide
tgttgagtgagggacaggatt

SEQ ID NO: 156 - DNA, 21 bp, Artificial Sequence
  Oligonulceotide
aacctcaaagctgcatcatca

SEQ ID NO: 157 - DNA, 21 bp, Artificial Sequence
  Oligonulceotide
cacaggtgaattctaacaggc

SEQ ID NO: 158 - DNA, 21 bp, Artificial Sequence
  Oligonulceotide
agtctaagctgaggcatggat

SEQ ID NO: 159 - DNA, 21 bp, Artificial Sequence
  Oligonulceotide
atgtaccaaggcaggctcttc

SEQ ID NO: 160 - DNA, 21 bp, Artificial Sequence
  Oligonulceotide
aatattaggatggtgtctgtc

SEQ ID NO: 161 - DNA, 21 bp, Artificial Sequence
  Oligonulceotide
tatagctggttgtgagggctc

SEQ ID NO: 162 - DNA, 21 bp, Artificial Sequence
  Oligonulceotide
tcattgagtgtcctctgcctc

SEQ ID NO: 163 - DNA, 21 bp, Artificial Sequence
  Oligonulceotide
actaacagctagaggctttgc

SEQ ID NO: 164 - DNA, 21 bp, Artificial Sequence
  Oligonulceotide
ccacttgcagactccatttgc

SEQ ID NO: 165 - DNA, 21 bp, Artificial Sequence
  Oligonulceotide
ataggtacacgtaggacttgc

SEQ ID NO: 166 - DNA, 21 bp, Artificial Sequence
  Oligonulceotide
atttcattgagtgtcctctgc

SEQ ID NO: 167 - DNA, 21 bp, Artificial Sequence
  Oligonulceotide
tagattgagacagagatgggc

SEQ ID NO: 168 - DNA, 21 bp, Artificial Sequence
  Oligonulceotide
tgactggatgtaagtataggc

SEQ ID NO: 169 - DNA, 21 bp, Artificial Sequence
  Oligonulceotide
tgttcaaggtatggagcaggc

SEQ ID NO: 170 - DNA, 21 bp, Artificial Sequence
  Oligonulceotide
agttcacgaatgtaccaaggc

SEQ ID NO: 171 - DNA, 21 bp, Artificial Sequence
  Oligonulceotide
tgttacgggaataggaggagc

SEQ ID NO: 172 - DNA, 21 bp, Artificial Sequence
  Oligonulceotide
ctaacagctagaggctttgcc

SEQ ID NO: 173 - DNA, 21 bp, Artificial Sequence
  Oligonulceotide
taggtacacgtaggacttgcc

SEQ ID NO: 174 - DNA, 21 bp, Artificial Sequence
  Oligonulceotide
tttcattgagtgtcctctgcc

SEQ ID NO: 175 - DNA, 21 bp, Artificial Sequence
  Oligonulceotide
atagttaccctggtttctacc

SEQ ID NO: 176 - DNA, 21 bp, Artificial Sequence
  Oligonulceotide
ttctaccagatcttgtcggac

SEQ ID NO: 177 - DNA, 21 bp, Artificial Sequence
  Oligonulceotide
ttctagcactctctttcattt

SEQ ID NO: 178 - DNA, 21 bp, Artificial Sequence
  Oligonulceotide
aagtataggcacgtgagtgtt

SEQ ID NO: 179 - DNA, 21 bp, Artificial Sequence
  Oligonulceotide
tcactactaggcagagctgtt

SEQ ID NO: 180 - DNA, 21 bp, Artificial Sequence
  Oligonulceotide
acaactaacagctagaggctt

SEQ ID NO: 181 - DNA, 21 bp, Artificial Sequence
  Oligonulceotide
acactcacgtgcctatactta

SEQ ID NO: 182 - DNA, 21 bp, Artificial Sequence
  Oligonulceotide
ttcatttggtatccgctttct

SEQ ID NO: 183 - DNA, 21 bp, Artificial Sequence
  Oligonulceotide
agtgagacgtggggtacatct

SEQ ID NO: 184 - DNA, 21 bp, Artificial Sequence
  Oligonulceotide
tggtatccgctttctcctgct

SEQ ID NO: 185 - DNA, 21 bp, Artificial Sequence
  Oligonulceotide
ttcattgagtgtcctctgcct

SEQ ID NO: 186 - DNA, 21 bp, Artificial Sequence
  Oligonulceotide
actagtaaagggtggtcttat

SEQ ID NO: 187 - DNA, 21 bp, Artificial Sequence
  Oligonulceotide
atagtgcccgtggttccagat

SEQ ID NO: 188 - DNA, 21 bp, Artificial Sequence
  Oligonulceotide
tgttctagcactctctttcat

SEQ ID NO: 189 - DNA, 21 bp, Artificial Sequence
  Oligonulceotide
tattaggatggtgtctgtcat

SEQ ID NO: 190 - DNA, 21 bp, Artificial Sequence
  Oligonulceotide
caatacctctctgccactttc

SEQ ID NO: 191 - DNA, 21 bp, Artificial Sequence
  Oligonulceotide
ctaagaaggatgatgctgttc

SEQ ID NO: 192 - DNA, 21 bp, Artificial Sequence
  Oligonulceotide
cactactaggcagagctgttc

SEQ ID NO: 193 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aguugctgctttaggtggagu

SEQ ID NO: 194 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
agaugaagccttggtcagugc

SEQ ID NO: 195 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uuucutgcggaactccagcuc

SEQ ID NO: 196 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
acaggtgaattctaacaggcc

SEQ ID NO: 197 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uaguucctctgcagtcuaagc

SEQ ID NO: 198 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
auaaatgagtagttccucugc

SEQ ID NO: 199 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uuugagtgtactgaggcaggc

SEQ ID NO: 200 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
guucaaggaagaagaggaggc

SEQ ID NO: 201 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aguugctgctttaggtggagu

SEQ ID NO: 202 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aaacagcacattcccagaguu

SEQ ID NO: 203 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uugugaaggctctgttuggcu

SEQ ID NO: 204 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
guugugaaggctctgtuuggc

SEQ ID NO: 205 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
guuguttgagtgtactgaggc

SEQ ID NO: 206 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aggugaattctaacaggccgu

SEQ ID NO: 207 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
gucuaagctgaggcatggauc

SEQ ID NO: 208 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
agucuaagctgaggcauggau

SEQ ID NO: 209 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
gaugatgccacagatgaagcc

SEQ ID NO: 210 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uguugagtgagggacaggauu

SEQ ID NO: 211 - DNA, 21 bp, Artificial Sequence
  Oligonulceotide
  misc_feature: Residues 1-5, 18-21 are FANA modified bases with Phosphorothioate Linkage; Residues 6-17 are bases with Phosphorothioate Linkage
aaccucaaagctgcatcauca

SEQ ID NO: 212 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
cacaggtgaattctaacaggc

SEQ ID NO: 213 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
agucuaagctgaggcauggau

SEQ ID NO: 214 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
auguaccaaggcaggcucuuc

SEQ ID NO: 215 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aauautaggatggtgtcuguc

SEQ ID NO: 216 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uauagctggttgtgagggcuc

SEQ ID NO: 217 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
ucauugagtgtcctctgccuc

SEQ ID NO: 218 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
acuaacagctagaggcuuugc

SEQ ID NO: 219 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
ccacutgcagactccauuugc

SEQ ID NO: 220 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
auaggtacacgtaggacuugc

SEQ ID NO: 221 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
auuucattgagtgtccucugc

SEQ ID NO: 222 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uagautgagacagagaugggc

SEQ ID NO: 223 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
ugacuggatgtaagtauaggc

SEQ ID NO: 224 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uguucaaggtatggagcaggc

SEQ ID NO: 225 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aguucacgaatgtaccaaggc

SEQ ID NO: 226 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uguuacgggaataggaggagc

SEQ ID NO: 227 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
cuaacagctagaggctuugcc

SEQ ID NO: 228 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uagguacacgtaggacuugcc

SEQ ID NO: 229 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uuucattgagtgtcctcugcc

SEQ ID NO: 230 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
auagutaccctggtttcuacc

SEQ ID NO: 231 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uucuaccagatcttgtcggac

SEQ ID NO: 232 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uucuagcactctctttcauuu

SEQ ID NO: 233 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aaguataggcacgtgaguguu

SEQ ID NO: 234 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
ucacuactaggcagagcuguu

SEQ ID NO: 235 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
acaactaacagctagaggcuu

SEQ ID NO: 236 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
acacucacgtgcctatacuua

SEQ ID NO: 237 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uucauttggtatccgcuuucu

SEQ ID NO: 238 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
agugagacgtggggtacaucu

SEQ ID NO: 239 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
ugguatccgctttctccugcu

SEQ ID NO: 240 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uucautgagtgtcctcugccu

SEQ ID NO: 241 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
acuagtaaagggtggtcuuau

SEQ ID NO: 242 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
auagugcccgtggttccagau

SEQ ID NO: 243 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uguuctagcactctctuucau

SEQ ID NO: 244 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
uauuaggatggtgtctgucau

SEQ ID NO: 245 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
caauacctctctgccacuuuc

SEQ ID NO: 246 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
cuaagaaggatgatgcuguuc

SEQ ID NO: 247 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
cacuactaggcagagcuguuc

SEQ ID NO: 248 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
agaugaagccttggtcagugc

SEQ ID NO: 249 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uuucuugcggaactccagcuc

SEQ ID NO: 250 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
acaggugaattctaacaggcc

SEQ ID NO: 251 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uaguucctctgcagtcuaagc

SEQ ID NO: 252 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
auaaaugagtagttccucugc

SEQ ID NO: 253 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uuugagtgtactgaggcaggc

SEQ ID NO: 254 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
guucaaggaagaagaggaggc

SEQ ID NO: 255 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
acugaccaaggcttcaucugu

SEQ ID NO: 256 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aaacagcacattcccagaguu

SEQ ID NO: 257 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uugugaaggctctgtuuggcu

SEQ ID NO: 258 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
guugugaaggctctguuuggc

SEQ ID NO: 259 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
guuguutgagtgtacugaggc

SEQ ID NO: 260 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aggugaattctaacaggccgu

SEQ ID NO: 261 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
gucuaagctgaggcauggauc

SEQ ID NO: 262 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
agucuaagctgaggcauggau

SEQ ID NO: 263 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
gaugaugccacagatgaagcc

SEQ ID NO: 264 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uguugagtgagggacaggauu

SEQ ID NO: 265 - DNA, 21 bp, Artificial Sequence
  Oligonulceotide
  misc_feature: Residues 1-6, 17-21 are FANA modified bases with Phosphorothioate Linkage; Residues 7-16 are bases with Phosphorothioate Linkage
aaccucaaagctgcatcauca

SEQ ID NO: 266 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
cacaggtgaattctaacaggc

SEQ ID NO: 267 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
agucuaagctgaggcauggau

SEQ ID NO: 268 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
auguaccaaggcaggcucuuc

SEQ ID NO: 269 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aauauuaggatggtgucuguc

SEQ ID NO: 270 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uauagctggttgtgagggcuc

SEQ ID NO: 271 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ucauugagtgtcctcugccuc

SEQ ID NO: 272 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
acuaacagctagaggcuuugc

SEQ ID NO: 273 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ccacuugcagactccauuugc

SEQ ID NO: 274 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
auagguacacgtaggacuugc

SEQ ID NO: 275 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
auuucattgagtgtccucugc

SEQ ID NO: 276 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uagauugagacagagaugggc

SEQ ID NO: 277 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ugacuggatgtaagtauaggc

SEQ ID NO: 278 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uguucaaggtatggagcaggc

SEQ ID NO: 279 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aguucacgaatgtaccaaggc

SEQ ID NO: 280 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uguuacgggaataggaggagc

SEQ ID NO: 281 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
cuaacagctagaggcuuugcc

SEQ ID NO: 282 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uagguacacgtaggacuugcc

SEQ ID NO: 283 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uuucautgagtgtccucugcc

SEQ ID NO: 284 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
auaguuaccctggttucuacc

SEQ ID NO: 285 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uucuaccagatcttgucggac

SEQ ID NO: 286 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uucuagcactctcttucauuu

SEQ ID NO: 287 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
aaguauaggcacgtgaguguu

SEQ ID NO: 288 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ucacuactaggcagagcuguu

SEQ ID NO: 289 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
acaacuaacagctagaggcuu

SEQ ID NO: 290 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
acacucacgtgcctauacuua

SEQ ID NO: 291 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uucauutggtatccgcuuucu

SEQ ID NO: 292 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
agugagacgtggggtacaucu

SEQ ID NO: 293 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
ugguauccgctttctccugcu

SEQ ID NO: 294 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uucauugagtgtcctcugccu

SEQ ID NO: 295 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
acuaguaaagggtggucuuau

SEQ ID NO: 296 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
auagugcccgtggttccagau

SEQ ID NO: 297 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uguucuagcactctcuuucau

SEQ ID NO: 298 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
uauuaggatggtgtcugucau

SEQ ID NO: 299 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
caauacctctctgccacuuuc

SEQ ID NO: 300 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
cuaagaaggatgatgcuguuc

SEQ ID NO: 301 - DNA, 21 bp, Artificial Sequence
  Oligonulceotide
  misc_feature: Residues 1-6, 16-20 are FANA modified bases with Phosphorothioate Linkage; Residues 7-15 are bases with Phosphorothioate Linkage; Residue 21 is FANA modified base
cacuactaggcagagcuguuc

SEQ ID NO: 302 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
gguugctgtctttcctgggug

SEQ ID NO: 303 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
gguugctgtctttcctgggug

SEQ ID NO: 304 - DNA, 21 bp, Artificial Sequence
  Oligonulceotide
  misc_feature: Residues 1-5, 17-20 are FANA modified bases with Phosphorothioate Linkage; Residues 6-16 are bases with Phosphorothioate Linkage; Residue 21 is FANA modified base
aguugctgctttaggtggagu

(19) United States
(12) Patent Application Publication
(10) Pub. No.: US 2021/0340536 A1
(43) Pub. Date: Nov. 4, 2021

(54) 2' FANA MODIFIED FOXP3 ANTISENSE OLIGONUCLEOTIDES AND METHODS OF USE THEREOF

(71) Applicants: AUM Lifetech, Inc., Philadelphia, PA (US); The Children's Hospital of Philadelphia, Philadelphia, PA (US)

(72) Inventors: Veenu Aishwarya, Philadelphia, PA (US); Wayne W. Hancock, Philadelphia, PA (US)

(21) Appl. No.: 17/279,542

(22) PCT Filed: Sep. 25, 2019
(86) PCT No.: PCT/US2019/053033

§ 371 Date: Mar. 24, 2021

Related U.S. Application Data
(60) Provisional application No. 62/739,001, filed on Sep. 28, 2018.
(60) Provisional application No. 62/737,061, filed on Sep. 26, 2018.

(51) Int. Cl. C12N 15/113; A61K 31/7088; A61K 45/06; A61P 35/00; A61N 5/10

(52) U.S. Cl. CPC C12N 15/113; A61K 31/7088; A61K 45/06; C12N 2310/322; A61N 5/10; C12N 2310/11; A61P 35/00

(57) ABSTRACT

The present invention is directed to hybrid chimera antisense oligonucleotides including deoxyribonucleotide and 2′-deoxy-2′-fluoro-β-D-arabinonucleotide which binds to a Foxp3 mRNA, and to methods of use thereof. The methods include the use for reducing expression level of Foxp3 gene, increasing anti-tumor activity, and treating cancer in a subject.

### CROSS-REFERENCE TO RELATED APPLICATIONS

This application claims benefit of priority under 35 U.S.C. § 119(e) of U.S. Ser. No. 62/737,061, filed Sep. 26, 2018, and U.S. Ser. No. 62/739,001, filed Sep. 28, 2018, the entire contents of both are incorporated herein by reference in its entirety.

### GOVERNMENT SUPPORT

This invention was made with government support under grants 5R01CA177852 and 5R01AI123241 awarded by the National Institutes of Health. The government has certain rights in the invention.

### INCORPORATION OF SEQUENCE LISTING

The material in the accompanying sequence listing is hereby incorporated by reference into this application. The accompanying sequence listing text file, name AUM1190_2WO_Sequence_Listing.txt, was created on ______, and is ______ kb. The file can be accessed using Microsoft Word on a computer that uses Windows OS.

### BACKGROUND OF THE INVENTION

### Field of the Invention

The present invention relates generally to hybrid chimera antisense oligonucleotides, and more specifically to the use of antisense oligonucleotides including deoxyribonucleotide and 2′-deoxy-2′-fluoro-β-D-arabinonucleotide for reducing expression level of Foxp3 gene, increasing anti-tumor activity, and treating cancer.

### Background Information

Cancer is a heterogeneous condition marked by unchecked cell growth and metastasis, leading to significant morbidity and mortality. Over a quarter of cancer-related deaths in both men and women are from lung cancer, which are estimated to be 154,050 in the United States in 2018. The current 5-year survival rate for all stages of lung cancer is 18.6%, despite advancements in surgical intervention, radiation, chemotherapy, and other treatments.

One of the reasons for this high mortality rate is the ability of tumors to evade destruction by the immune system. Successful infiltration of CD8⁺ T cells into the tumor microenvironment has been shown to improve outcomes, while infiltration by FOXP3⁺CD4⁺CD25⁺ regulatory T cells (Treg cells) have been shown to correlate with negative clinical outcomes in various types of cancer. Treg cells actively suppress anti-tumor activity by T effector cells, B cells, NK cells, macrophages, and dendritic cells. This prevents the body from attacking the cancerous cells and/or tumor, and so worsens prognosis. Treg function and immune suppression is dependent upon FOXP3, and there are currently no effective ways to target these cells.

Single-stranded synthetic oligonucleotides, called antisense oligonucleotides (ASOs or AONs) are one means of nucleic acid therapeutics. They recognize sequences of target RNA and can achieve gene silencing. There are several potential mechanisms by which this occurs, one of which being RNase H-mediated cleavage of the target RNA once it is bound to an AON. While conventional AONs have been effective in discovery and preclinical studies, their translation to the clinic has been plagued by a number of challenges, including target accessibility, off-targeting effects, poor stability, and poor delivery to target cells.

There is currently an unmet need for new therapeutics using next generation AON chemistries to reduce Treg immune suppression and promote anti-tumor immunity, especially in lung cancer.

### SUMMARY OF THE INVENTION

The present invention is based on the seminal discovery that a hybrid chimera antisense oligonucleotide including deoxyribonucleotide and 2′-deoxy-2′-fluoro-β-D-arabinonucleotide, which binds to a Foxp3 mRNA, can be used for reducing expression level of Foxp3 gene, increasing anti-tumor activity, and treating cancer.

In one embodiment, the present invention provides a modified antisense oligonucleotide (AON) including at least one 2′-deoxy-2′-fluoro-β-D-arabinonucleotide (2′-FANA modified nucleotide), wherein the AON binds to a Foxp3 mRNA.

In various aspects, 2′-FANA modified nucleotides are positioned according to any of Formulas 1-16. In one aspect, the 2′-FANA modified nucleotides are positioned according to Formula 6. In certain aspects, the internucleotide linkages between nucleotides of the 2′-FANA modified nucleotides are phosphodiester bonds, phosphotriester bonds, phosphorothioate bonds (5′O—P(S)O—3O—, 5′S—P(O)O—3′—O—, and 5′O—P(O)O—3′S—), phosphorodithioate bonds, Rp-phosphorothioate bonds, Sp-phosphorothioate bonds, boranophosphate bonds, methylene bonds (methylimino), amide bonds (3′—CH2—CO—NH—5′ and 3′—CH2—NH—CO—5′), methylphosphonate bonds, 3′-thioformacetal bonds, (3′S—CH2—O5′), amide bonds (3′CH2—C(O)NH—5′), phosphoramidate groups, or a combination thereof.

In various aspects, the AON is a hybrid chimera AON including at least one 2′-FANA modified nucleotide and at least one unmodified deoxyribonucleotide, wherein the AON is a 2′-FANA AON. In some aspects, the 2′-FANA AON includes from about 0 to about 20 2′-deoxy-2′-fluoro-β-D-arabinonucleotides at the 5′-end and from about 0 to about 20 2′-deoxy-2′-fluoro-β-D-arabinonucleotides at the 3′-end, flanking a sequence including from about 0 to about 20 deoxyribonucleotide residues. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID NOs: 193-302, or a sequence complimentary thereto.

In another embodiment, the invention provides a pharmaceutical composition including a modified antisense oligonucleotide (AON) including at least one 2′-deoxy-2′-fluoro-β-D-arabinonucleotide (2′-FANA modified nucleotide) and a pharmaceutically acceptable carrier, wherein the AON binds to a Foxp3 mRNA.

In various aspects, the AON is a hybrid chimera AON including at least one 2′-FANA modified nucleotide and at least one unmodified deoxyribonucleotide, wherein the AON is a 2′-FANA AON. In some aspects, the 2′-FANA AON includes from about 0 to about 20 2′-deoxy-2′-fluoro-β-D-arabinonucleotides at the 5′-end and from about 0 to about 20 2′-deoxy-2′-fluoro-β-D-arabinonucleotides at the 3′-end, flanking a sequence including from about 0 to about 20 deoxyribonucleotide residues. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID NOs: 193-302, or a sequence complimentary thereto.

In an additional embodiment, the invention provides a method of reducing the expression level of Foxp3 gene in a cell including contacting the cell with at least one antisense oligonucleotide (AON), wherein the AON binds to Foxp3 mRNA, and wherein the AON includes at least one 2′-deoxy-2′-fluoro-β-D-arabinonucleotide (2′-FANA modified nucleotide).

In one aspect, the cell is a regulatory T cell (Treg). In various aspects, the Treg expresses the cellular markers CD4 and CD25.

In other aspects, the AON is a hybrid chimera AON including and at least one 2′-FANA modified nucleotide and at least one unmodified deoxyribonucleotide, wherein the AON is a 2′-FANA AON. In various aspects, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID Nos: 139-192, SEQ ID NOs: 193-302, or a sequence complimentary thereto.

In another embodiment, the invention provides a method of increasing anti-tumor immunity in a subject in need thereof including administering to the subject at least one antisense oligonucleotide (AON), wherein the AON binds to a Foxp3 mRNA, and wherein the AON includes at least one 2′-deoxy-2′-fluoro-β-D-arabinonucleotide (2′-FANA modified nucleotide).

In one aspect, the AON decreases the activity of a regulatory T cell (Treg). In some aspects, the Treg expresses the cellular markers CD4 and CD25. In various aspects, the AON induces Treg apoptosis. In another aspect, the AON increases the activity of an immune cell. In certain aspects, the immune cell is CD8⁺ T cell, CD4⁺ T cell, B cell, Natural Killer cell, macrophage, dendritic cell or a combination thereof.

In various aspects, the AON is a hybrid chimera AON including at least one 2′-FANA modified nucleotide and at least one unmodified deoxyribonucleotide, wherein the AON is a 2′-FANA AON. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID Nos: 193-302, or a sequence complimentary thereto.

In yet another embodiment, the invention provides a method of treating cancer in a subject in need thereof including administering to the subject at least one antisense oligonucleotide (AON), wherein the AON binds to a Foxp3 mRNA, and wherein the AON includes at least one 2′-deoxy-2′-fluoro-β-D-arabinonucleotide (2′-FANA modified nucleotide).

In various aspects, the AON reduces expression level of a Foxp3 gene. In some aspects, the AON is a hybrid chimera AON including at least one 2′-FANA modified nucleotide and at least one unmodified deoxyribonucleotide, wherein the AON is a 2′-FANA AON. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs 21-29, SEQ ID NOs: 31-138, SEQ ID NOs: 139-192, SEQ ID NOs: 193-302, or a sequence complimentary thereto.

In various aspects, the 2′-FANA AON increases anti-tumor immunity in the subject. In some aspects, the 2′-FANA AON decreases the activity of a regulatory T cell (Treg) and/or increases the activity of an immune cell.

In certain aspects, the AON further includes a pharmaceutically acceptable carrier. In other aspects, an immunotherapeutic agent and/or a chemotherapeutic agent is further administered. In certain aspects, the immunotherapeutic agent and/or chemotherapeutic agent is a checkpoint inhibitor, vaccine, chimeric antigen receptor (CAR)-T cell therapy, anti-PD-1 antibody (Nivolumab or Pembrolizumab), anti-PD-L1 antibody (Atezolizumab, Avelumab or Durvalumab) or a combination thereof. In other aspects, the immunotherapeutic agent and/or chemotherapeutic agent is administered prior to, simultaneously with, or after the administration of the AON. In certain aspects, a radiotherapy is further administered. In certain aspects, the radiotherapy is administered prior to, simultaneously with, or after the administration of the AON.

In certain aspects, the cancer is breast, liver, ovarian, pancreatic, lung cancer, melanoma or glioblastoma. In one aspect, the cancer is lung cancer.

### BRIEF DESCRIPTION OF THE DRAWINGS

FIG. 1 shows flow cytometry dot plots illustrating the number of Foxp3 expressing cells in a splenocyte cell population. Scramble: control FANA antisense oligonucleotide; Foxp3-1 to 9: nine different FANA oligonucleotides targeting Foxp3; 2.5 uM and 5 uM: concentrations of Foxp3 FANA; Fluoro: negative control, auto-fluorescence of the cells.

FIG. 2 shows flow cytometry dot plots illustrating the number of Foxp3 expressing cells in a purified Treg cell population. Scramble: control FANA antisense oligonucleotide; Foxp3-1 to 9: nine different FANA oligonucleotides targeting Foxp3; 2.5 uM and 5 uM: concentrations of Foxp3 FANA.

FIG. 3 shows flow cytometry dot plots illustrating the in vivo effect Foxp3 FANAs on the number of Foxp3 expressing cells.

FIG. 4 shows flow cytometry dot plots illustrating the in vivo cellular uptake of APC labeled FANA by spleen, lymph node and blood cells. IV: intravenous.

FIG. 5 shows flow cytometry dot plots illustrating the in vivo cellular uptake of labeled FANA by non-Treg Foxp3+ cells of spleen, lymph node and blood. IV: intravenous.

FIG. 6 shows flow cytometry dot plots illustrating the in vivo cellular uptake of labeled FANA by Treg cells of spleen, lymph node and blood. IV: intravenous.

FIG. 7 shows confocal microscopy image illustrating the uptake of FANA antisense oligonucleotide by Foxp3 expressing cells. N: nucleus, *: FANA; arrowhead: Foxp3.

FIGS. 8A-8B show the in vitro effect of Foxp3-FANA on the protein expression level of Foxp3 measured by western blot. FIG. 8A are immunoblots showing Foxp3 expression; FIG. 8B illustrates Foxp3 quantification.

FIGS. 9A-9B show the in vivo effect of Foxp3 FANAs on the protein expression level of Foxp3 measured by western blot. B6: untreated control. FIG. 9A are immunoblots showing Foxp3 expression; FIG. 9B illustrates Foxp3 quantification.

FIG. 10 shows histograms illustrating Treg immune suppression assay.

FIG. 11 shows flow cytometry dot plots illustrating the in vivo effect of Foxp3 FANAs on Treg suppressive function.*: significant difference as compared to the control.

FIGS. 12A-12B show the in vivo effect of Foxp3 FANAs on the protein expression level of Foxp3 measured by western blot. FIG. 12A are immunoblots showing Foxp3 expressions; FIG. 12B illustrates Foxp3 quantification.

FIG. 13 shows growth curves illustrating the in vivo effect of Foxp3 FANAs on tumor growth.

FIGS. 14A-14C show the in vivo effect of AUM-FANA-6 the growth of TC1 lung tumor in mice. FIG. 14A shows growth curves of illustrating tumor volumes in control and AUM-FANA-6 (SEQ ID NO:26) treated mice; FIG. 14B shows individual growth curves illustrating in vivo effect of Foxp3 AUM-FANA-6 (SEQ ID NO:304) on tumor growth; FIG. 14C shows the number of Foxp3⁺ CD4⁺ cells.

FIGS. 15A-15B show the in vivo effect of Foxp3 FANAs on the number of intratumoral Foxp3⁺ Treg cells measured by flow cytometry. FIG. 15A shows flow cytometry dot plots; FIG. 15B illustrates the Foxp3⁺ CD4⁺ intratumoral cells quantification.

FIGS. 16A-16B show the in vivo effect of Foxp3 FANAs on the number of intrasplenic Foxp3⁺ cells measured by flow cytometry. FIG. 16A shows flow cytometry dot plots; FIG. 16B illustrates the Foxp3⁺ CD4⁺ intrasplenic cells quantification.

FIG. 17 shows an immunoblot illustrating the in vitro knockdown of Foxp3 after treatment with 0.1 or 0.5 μM of AUM-FANA-6 (SEQ ID NO:26).

FIG. 18 shows flow cytometry dot plots illustrating the in vitro effect of nine different FANAs on the population of Foxp3⁺ Tregs in murine splenocytes. Using either 2.5 or 5 μM of FANAs oligonucleotides.

FIGS. 19A-19B show the in vitro effect of AUM-FANA-5 (SEQ ID NO:25), AUM-FANA-5B (SEQ ID NO: 303), AUM-FANA-6 (SEQ ID NO:26) and AUM-FANA-6B on Treg suppressive function. FIG. 19A shows histograms illustrating Treg proliferation; FIG. 19B illustrates the quantification of dividing cells.

FIG. 20 shows immunoblots illustrating the in vivo effect of Foxp3 FANA on Foxp3 expression in draining lymph nodes of tumor-bearing mice.

FIGS. 21A-21B show the in vivo effect of Foxp3 AUM-FANA-6B (SEQ ID NO: 304) in lung tumor growth in mice. FIG. 21A shows tumor growth over time; FIG. 21B is a graph bar illustrating the quantification at the end of the experiment.

FIG. 22 shows the in vivo effect of Foxp3 FANA on anti-tumor immunity in a mice model of lung cancer. LN: lymph node; SP: spleen, T: tumor.

### DETAILED DESCRIPTION OF THE INVENTION

The present invention is based on the seminal discovery that hybrid chimera antisense oligonucleotides including deoxyribonucleotide and 2′-deoxy-2′-fluoro-β-D-arabinonucleotide, which binds to a Foxp3 mRNA, can be used for reducing expression level of Foxp3 gene, for increasing anti-tumor activity, and for treating cancer.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a modified antisense oligonucleotide (AON) including at least one 2′-deoxy-2′-fluoro-β-D-arabinonucleotide (2′-FANA modified nucleotide), wherein the AON binds to a Foxp3 mRNA.

As used herein “modified antisense oligonucleotide” refers to synthetic antisense oligonucleotides (AONs) containing modified sugar. AONs are single stranded oligonucleotides that recognize nucleic acid sequences via Watson-Crick base pairing and cause pre- or post-transcriptional gene silencing. The AON binds to its target mRNA, and forms a duplex that is recognized by RNase H, which in turn induces the cleavage of the mRNA, the steric blocking of translation machinery, or the prevention of necessary RNA interactions.

Sugar-modified oligonucleotides are well known in the art (see U.S. Pat. No. 8,178,348, and US Patent Application Nos 2005/0233455, 2009/015467, and 2005/0142535 for example). These nuclease resistant oligonucleotides can form duplexes with DNA and RNA sequences, and thus inhibit gene expression. Several types of analogues have been described, wherein changes in the sugar, the sugar backbone, or the internucleotide linkage for example were assessed as ways of modulating enzymatic stability, duplex forming capability, or RNaseH recruitment, aiming at designing clinically relevant molecules capable of forming more stable complexes, for which RNaseH has a strong affinity, resulting in more efficient gene silencing.

Among the analogues, mixed back-bone oligonucleotides (MBO) including phosphodiester and phosphorothiotate oligonucleotides to make them more suitable substrates for RNaseH; oligonucleotides containing hexopyranose instead of pentofurase, such as peptide nucleic acids (PNA) which include an acyclic backbone and have generally increased enzymatic stability but reduced duplex forming capability; and arabinonucleosides as used herein and further described hereinafter have been described and synthesized.

Additionally, chimera oligonucleotides, comprising modified nucleosides alternating with unmodified nucleoside have also been described, and are known for their strong impact on gene expression in cells and organism.

Chemical strategies are known to improve nucleotide stability, which include modification of the ribose sugar moiety, the phosphodiester backbone, and the bases. The phosphodiester backbone in particular is often replaced with phosphorothioate (PS) backbone. The PS backbone is made when one of the non-bridging atoms in the backbone is replaced with a sulfur. For example, a modification made to the 2′ position of the ribose sugar results in arabinonucleosides, such as 2′-deoxy-2′-fluoroarabinonucleotide (2′-FANA)-modified nucleotide, as used herein.

Foxp3, also known as forkhead box P3 or scurfin, is a protein involved in immune system responses. As a member of the FOX protein family, Foxp3 appears to function as a master regulator of the regulatory pathway in the development and function of regulatory T cells. While the precise control mechanism has not yet been established, FOX proteins belong to the forkhead/winged-helix family of transcriptional regulators and are presumed to exert control via similar DNA binding interactions during transcription. In regulatory T cell model systems, the FOXP3 transcription factor occupies the promoters for genes involved in regulatory T-cell function, and may repress transcription of key genes following stimulation of T cell receptors.

Foxp3 is a specific marker of natural T regulatory cells (nTregs), a lineage of T cells and adaptive/induced T regulatory cells (a/iTregs), also identified by other less specific markers such as CD25 or CD45RB. In animal studies, Tregs that express Foxp3 are critical in the transfer of immune tolerance, especially self-tolerance.

In various aspects, the AON is a hybrid chimera AON including at least one 2′-FANA modified nucleotide and at least one unmodified deoxyribonucleotide, wherein the AON is a 2′-FANA AON.

In certain embodiments, the modified AON of the invention includes at least one 2′-FANA modified nucleotide. In various embodiments, the modified AON includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 2′-FANA modified nucleotides. In other embodiments, the modified AON of the invention includes at least 2, at least 3, at least 4, at least 5, at least 6, at least 7, at least 8, at least 9,at least 10, at least 11, at least 12, at least 13, at least 14, at least 15, at least 16, at least 17, at least 18, at least 19, at least 20, at least 21, at least 22, at least 23, at least 24, or at least 25 2′-FANA modified nucleotides.

“Naturally occurring nucleotide” or “unmodified nucleotides” contain normally occurring sugars (D-ribose and D-2-deoxyribose) and a phosphodiester backbone that are readily degraded by nucleases. As used herein, unmodified nucleotides are referred to as deoxyribonucleotide.

As used herein a “2′-FANA modified AON” or “2′-FANA AON”, or “Foxp3 FANA” and the like, refers to AONs that target a portion of Foxp3 mRNA. 2′-FANA AON are designed to target a portion of the Foxp3 mRNA expression. 2′-FANA AON results from the incorporation of at least one 2′-FANA modified nucleotide in an antisense oligonucleotide. The modified synthetic oligonucleotides described herein include at least one nucleotide that has a 2′-FANA modification, and so are 2′-FANA oligonucleotides (2′-FANA AONs or 2′-FANA ASOs). These modified synthetic oligonucleotides can include a phosphorothioate backbone. They can also include other backbone modifications, which are discussed later in this disclosure. The incorporation of 2′-FANA nucleotides confers high stability, specificity, and affinity for the target. 2′-FANA ASOs are also capable of self-delivery, which negates the need for a delivery agent. The lack of a delivery agent reduces toxicity in various models. Thus, in some embodiments, at least a portion of the 2′-FANA AON is complementary to part of an mRNA sequence that corresponds to the Foxp3 gene. The 2′-FANA AON may be designed to target and bind to all or a portion of the Foxp3 mRNA. In some embodiments, a synthetic AON comprising a 2′-FANA modified sequence according to any embodiment described herein inhibits expression of Foxp3. In certain embodiments, the 2′FANA modified AON described herein inhibits expression of Foxp3 cells by binding to a portion of the mRNA and triggering cleavage by RNase H.

The chemistry and construction of 2′FANA oligonucleotides has been described elsewhere in detail (See, e.g., U.S. Pat. Nos. 8,278,103 and 9,902,953, each of which is specifically incorporated herein in their entirety by reference). The 2′-FANA AONs and methods of using them disclosed herein contemplate any FANA chemistries known in the art. In some embodiments, a 2′-FANA AON comprises an internucleoside linkage comprising a phosphate, thereby being an oligonucleotide. In some embodiments, the sugar-modified nucleosides and/or 2′-deoxynucleosides comprise a phosphate, thereby being sugar-modified nucleotides and/or 2′-deoxynucleotides. In some embodiments, a 2′-FANA AON comprises an internucleoside linkage comprising a phosphorothioate. In some embodiments, the internucleoside linkage is selected from phosphorothioate, phosphorodithioate, methylphosphorothioate, Rp-phosphorothioate, Sp-phosphorothioate. In some embodiments, the a 2′-FANA AON comprises one or more internucleotide linkages selected from the group consisting of: (a) phosphodiester; (b) phosphotriester; (c) phosphorothioate; (d) phosphorodithioate; (e) Rp-phosphorothioate; (f) Sp-phosphorothioate; (g) boranophosphate; (h) methylene (methylimino) (3′ CH2—N(CH3)—O5′); (i) 3′-thioformacetal (3′S—CH2—O5′); (j) amide (3′CH2—C(O)NH—5′); (k) methylphosphonate; (l) phosphoramidate (3′—OP(O2)—N5′); and (m) any combination of (a) to (l).

In some embodiments, 2′-FANA AONs comprising alternating segments or units of sugar-modified nucleotides (e.g., arabinonucleotide analogues [e.g., 2′-FANA]) and 2′-deoxyribonucleotides (DNA) are utilized. In some embodiments, a 2′-FANA AON disclosed herein comprises at least 2 of each of sugar-modified nucleotide and 2′-deoxynucleotide segments, thereby having at least 4 alternating segments overall. Each alternating segment or unit may independently contain 1 or a plurality of nucleotides. In some embodiments, each alternating segment or unit may independently contain 1 or 2 nucleotides. In some embodiments, the segments each comprise 1 nucleotide. In some embodiments, the segments each comprise 2 nucleotides. In some embodiments, the plurality of nucleotides may consist of 2, 3, 4, 5 or 6 nucleotides. A 2′-FANA AON may contain an odd or even number of alternating segments or units and may commence and/or terminate with a segment containing sugar-modified nucleotide residues or DNA residues. Thus, a 2′-FANA AON may be represented as follows:

A1-D1-A2-D2-A3-D3 . . . Az-Dz,

where each of A1, A2, etc. represents a unit of one or more (e.g., 1 or 2) sugar-modified nucleotide residues (e.g., 2′-FANA) and each of D1, D2, etc. represents a unit of one or more (e.g., 1 or 2) DNA residues. The number of residues within each unit may be the same or variable from one unit to another. The oligonucleotide may have an odd or an even number of units. The oligonucleotide may start (i.e. at its 5′ end) with either a sugar-modified nucleotide-containing unit (e.g., a 2′-FANA-containing unit) or a DNA-containing unit. The oligonucleotide may terminate (i.e. at its 3′ end) with either a sugar-modified nucleotide-containing unit or a DNA-containing unit. The total number of units may be as few as 4 (i.e. at least 2 of each type).

In some embodiments, a 2′-FANA AON disclosed herein comprises alternating segments or units of arabinonucleotides and 2′-deoxynucleotides, wherein the segments or units each independently comprise at least one arabinonucleotide or 2′-deoxynucleotide, respectively. In some embodiments, the segments each independently comprise 1 to 2 arabinonucleotides or 2′-deoxynucleotides. In some embodiments, the segments each independently comprise 2 to 5 or 3 to 4 arabinonucleotides or 2′-deoxynucleotides. In some embodiments, a 2′-FANA AON disclosed herein comprises alternating segments or units of arabinonucleotides and 2′-deoxynucleotides, wherein the segments or units each comprise one arabinonucleotide or 2′-deoxynucleotide, respectively. In some embodiments, the segments each independently comprise about 3 arabinonucleotides or 2′-deoxynucleotides. In some embodiments, a 2′-FANA AON disclosed herein comprises alternating segments or units of arabinonucleotides and 2′-deoxynucleotides, wherein the segments or units each comprise one arabinonucleotide or 2′-deoxynucleotide, respectively. In some embodiments, a 2′-FANA AON disclosed herein comprises alternating segments or units of arabinonucleotides and 2′-deoxynucleotides, wherein said segments or units each comprise two arabinonucleotides or 2′-deoxynucleotides, respectively.

In some embodiments, a 2′-FANA AON disclosed herein has a structure selected from the group consisting of:
a) (Ax-Dy)n I
b) (Dy-Ax)n II
c) (Ax-Dy)m-Ax-Dy-Ax III
d) (Dy-Ax)m-Dy-Ax-Dy IV
   wherein each of m, x and y are each independently an integer greater than or equal to 1, n is an integer greater than or equal to 2, A is a sugar-modified nucleotide and D is a 2′-deoxyribonucleotide.

For example, a 2′-FANA AON disclosed herein has structure I wherein x=1, y=1 and n=10, thereby having a structure:

A-D-A-D-A-D-A-D-A-D-A-D-A-D-A-D-A-D-A-D.

In another example, a 2′-FANA AON disclosed herein has structure II wherein x=1, y=1 and n=10, thereby having a structure:

D-A-D-A-D-A-D-A-D-A-D-A-D-A-D-A-D-A-D-A.

In another example, a 2′-FANA AON disclosed herein has structure III wherein x=1, y=1 and n=9, thereby having a structure:

A-D-A-D-A-D-A-D-A-D-A-D-A-D-A-D-A-D-A-D-A.

In another example, a 2′-FANA AON disclosed herein has structure IV wherein x=1, y=1 and n=9, thereby having a structure:

D-A-D-A-D-A-D-A-D-A-D-A-D-A-D-A-D-A-D-A-D.

In another example, a 2′-FANA AON disclosed herein has structure I wherein x=2, y=2 and n=5, thereby having a structure:

A-A-D-D-A-A-D-D-A-A-D-D-A-A-D-D-A-A-D-D.

In another example, a 2′-FANA AON disclosed herein has structure II wherein x=2, y=2 and n=5, thereby having a structure:

D-D-A-A-D-D-A-A-D-D-A-A-D-D-A-A-D-D-A-A.

In another example, a 2′-FANA AON disclosed herein has structure III wherein x=2, y=2 and m=4, thereby having a structure:

A-A-D-D-A-A-D-D-A-A-D-D-A-A-D-D-A-A-D-D-A-A.

In another example, a 2′-FANA AON disclosed herein has structure IV wherein x=2, y=2 and m=4, thereby having a structure:

D-D-A-A-D-D-A-A-D-D-A-A-D-D-A-A-D-D-A-A-D-D.

In various aspects, 2′-FANA modified nucleotides are positioned according to any of Formulas 1-16. In one aspect, the 2′-FANA modified nucleotides are positioned according to Formula 6.

The modified 2′-FANA AON sequence may include a modified sugar moiety for all or only a portion of the nucleotides in the sequence. In some embodiments, the AONs may have all modified sugar moiety nucleotides in the sequence. In some embodiments, the AONs may be between 1 and 60 nucleotides long. In some embodiments, the AONs may have 1, 2, 3, 4, 5, 6, 7, 8, 9, 10, 11, 12, 13, 14, 15, 16, 17, 18, 19, 20, or more than 20 unmodified nucleotides.

In some embodiments, at least one unmodified nucleotide is located in the AON between strings of nucleotides which have modified sugar moieties. For example, a modified AON may have a string of at least 2, at least 3, at least 4, at least 5, at least 6, at least 7, at least 8, at least 9, at least 10 or more 2′-FANA-modified nucleotides, followed by a string of at least 1, at least 2, at least 3, at least 4, at least 5, at least 6, at least 7, at least 8, at least 9, at least 10, or more unmodified nucleotides, followed by another string of at least 2, at least 3, at least 4, at least 5, at least 6, at least 7, at least 8, at least 9, at least 10 or more 2′-FANA-modified nucleotides. In certain embodiments, when one or more unmodified nucleotides are flanked by 2′FANA-modified nucleotides, the unmodified nucleotide section may be referred to as a “nucleotide gap sequence.” The gap sequence may consist of 1, 2, 3, 4, 5, 6, 7, 8, 9, 10, 11, 12, 13, 14, 15, 16, 17, 18, 19, 20, or more than 20 unmodified nucleotides. In some embodiments, the AON may have a single gap sequence or may have more than one nucleotide gap sequence in the same molecule. The string of 2′-FANA modified nucleotides on each side of the unmodified nucleotide gap sequence can be of the same or of different lengths. For example, the AON may have 8 2′-FANA modified nucleotides, followed by 7 unmodified nucleotides, followed by a second string of 2′-FANA modified nucleotides that is the same or different in number of 2′-FANA modified nucleotides as the first modified string. In certain embodiments the AON consists of 2′-FANA sugar modified nucleotides sequences flanking a gap sequence of unmodified nucleotides. For example, the AON comprises a 2′-FANA modified sequence between 1 and 10 nucleotides in length, then an unmodified nucleotide sequence between 1 and 10 nucleotides in length, followed by another 2′-FANA modified sequence between 1 and 10 nucleotides in length, with this pattern of modified and unmodified nucleotides optionally repeating. Table 1 illustrates exemplary dispositions of unmodified nucleotides and 2′-FANA modified nucleotides in 21-long oligonucleotides.

TABLE 1
Exemplary 2′-FANA nucleoside placement within 21-mer 2′-FANA AON

| 21 nucleotides | Formula |
|---|---|
| Formula 1 | XXXXXXXXXXXXXXXXXXXXX |
| Formula 2 | XXXXXXXXXXXXXXXXXXXXX |
| Formula 3 | XXXXXXXXXXXXXXXXXXXXX |
| Formula 4 | XXXXXXXXXXXXXXXXXXXXX |
| Formula 5 | XXXXXXXXXXXXXXXXXXXXX |
| Formula 6 | XXXXXXXXXXXXXXXXXXXXX |
| Formula 7 | XXXXXXXXXXXXXXXXXXXXX |
| Formula 8 | XXXXXXXXXXXXXXXXXXXXX |
| Formula 9 | XXXXXXXXXXXXXXXXXXXXX |
| Formula 10 | XXXXXXXXXXXXXXXXXXXXX |
| Formula 11 | XXXXXXXXXXXXXXXXXXXXX |
| Formula 12 | XXXXXXXXXXXXXXXXXXXXX |
| Formula 13 | XXXXXXXXXXXXXXXXXXXXX |
| Formula 14 | XXXXXXXXXXXXXXXXXXXXX |
| Formula 15 | XXXXXXXXXXXXXXXXXXXXX |
| Formula 16 | XXXXXXXXXXXXXXXXXXXXX |

The formulas shown in Table 1 can be applied to any sequence shown in SEQ ID Nos 1-9, 31-138, 11-19 139-192, or a portion thereof, wherein X represents a nucleotide (A, C, G, T, or U) and bolded and underlined nucleotides represent 2′-FANA modified nucleotides.

In certain aspects, the internucleotide linkages between nucleotides of the 2′-FANA modified nucleotides are phosphodiester bonds, phosphotriester bonds, phosphorothioate bonds (5′O—P(S)O—3O—, 5′S—P(O)O—3′—O—, and 5′O—P(O)O—3′S—), phosphorodithioate bonds, Rp-phosphorothioate bonds, Sp-phosphorothioate bonds, boranophosphate bonds, methylene bonds (methylimino), amide bonds (3′—CH2—CO—NH—5′ and 3′—CH2—NH—CO—5′), methylphosphonate bonds, 3′-thioformacetal bonds, (3′S—CH2—O5′), amide bonds (3′CH2—C(O)NH—5′), phosphoramidate groups, or a combination thereof.

In some aspects, the 2′-FANA AON includes from about 0 to about 20 2′-deoxy-2′-fluoro-β-D-arabinonucleotide at the 5′-end and from about 0 to about 20 2′-deoxy-2′-fluoro-β-D-arabinonucleotide at the 3′-end, flanking a sequence including from about 0 to about 20 deoxyribonucleotide residues. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID NOs: 193-302, any sequence from Table 2 or a sequence complimentary thereto.

The 2′-FANA AON of the invention comprises at least 5 successive nucleotides of SEQ ID NOs 1-9, SEQ ID NOs:11-19, SEQ ID NOs: 21-29, SEQ ID NOs: 31-138, SEQ ID NOs: 139-192, or SEQ ID NOs: 193-302. In some embodiments, the plurality of nucleotides comprises any one of the nucleotide sequence of SEQ ID NOs 1-9, SEQ ID NOs:11-19, SEQ ID NOs: 21-29, SEQ ID NOs: 31-138, SEQ ID NOs: 139-192, or SEQ ID NOs: 193-302 (Table 2) or an equivalent of each thereof For purposes of this disclosure, a molecule having a thymine or uracil at the same position is deemed equivalent of the following sequences. In some embodiments, the modified synthetic 2′-FANA AON has at least 60%, at least 65%, at least 70%, at least 75%, at least 80%, at least 85%, at least 90%, at least 95%, at least 98%, or at least 99% sequence identity to any one of the SEQ ID NOs 1-9, SEQ ID NOs:11-19, SEQ ID NOs: 21-29, SEQ ID NOs: 31-138, SEQ ID NOs: 139-192, or SEQ ID NOs: 193-302.

TABLE 2
2′-FANA oligonucleotides sequences

| SEQ ID NO: | Description | Sequence (from 5′-end to 3′-end) |
|---|---|---|
| SEQ ID NO: 1 | AUM-F-FXP3-1 | UCAUUGAGTGTCCTCUGCCUC |
| SEQ ID NO: 2 | AUM-F-FXP3-2 | UAGGUACACGTAGGACUUGCC |
| SEQ ID NO: 3 | AUM-F-FXP3-3 | AAUAUUAGGATGGTGUCUGUC |
| SEQ ID NO: 4 | AUM-F-FXP3-4 | GAAGUUGCCGGGAGAGCUGAA |
| SEQ ID NO: 5 | AUM-F-FXP3-5 | GGUUGCTGTCTTTCCUGGGUG |
| SEQ ID NO: 6 | AUM-F-FXP3-6 | AGUUGCTGCTTTAGGUGGAGU |
| SEQ ID NO: 7 | AUM-F-FXP3-7 | GAUGUGACTGTCTTCCAAGUC |
| SEQ ID NO: 8 | AUM-F-FXP3-8 | UGAAUUCAGTACTGCAGAGCU |
| SEQ ID NO: 9 | AUM-F-FXP3-9 | CUUUGACTGGAAAGTUCAUCU |
| SEQ ID NO: 10 | Scramble Control | UGACCCUATGCTGTUCCUAUA |
| SEQ ID NO: 11 | AUM-FXP3-1 | TCATTGAGTGTCCTCTGCCTC |
| SEQ ID NO: 12 | AUM-FXP3-2 | TAGGTACACGTAGGACTTGCC |
| SEQ ID NO: 13 | AUM-FXP3-3 | AATATTAGGATGGTGTCTGTC |
| SEQ ID NO: 14 | AUM-FXP3-4 | GAAGTTGCCGGGAGAGCTGAA |
| SEQ ID NO: 15 | AUM-FXP3-5 | GGTTGCTGTCTTTCCTGGGTG |
| SEQ ID NO: 16 | AUM-FXP3-6 | AGTTGCTGCTTTAGGTGGAGT |
| SEQ ID NO: 17 | AUM-FXP3-7 | GATGTGACTGTCTTCCAAGTC |
| SEQ ID NO: 18 | AUM-FXP3-8 | TGAATTCAGTACTGCAGAGCT |
| SEQ ID NO: 19 | AUM-FXP3-9 | CTTTGACTGGAAAGTTCATCT |
| SEQ ID NO: 20 | Scramble Control | TGACCCTATGCTGTTCCTATA |
| SEQ ID NO: 21 | AUM-FANA-FXP3-1 | [FANA U]*[FANA C]*[FANA A]*[FANA U]*[FANA |
| | | U]*[FANA G]*A*G*T*G*T*C*C*T*C*[FANA |
| | | U]*[FANA G]*[FANA C]*[FANA C]*[FANA |
| | | U]*[FANA C] |
| SEQ ID NO: 22 | AUM-FANA-FXP3-2 | [FANA U]*[FANA A]*[FANA G]*[FANA G]*[FANA |
| | | U]*[FANA A]*C*A*C*G*T*A*G*G*A*[FANA |
| | | C]*[FANA U]*[FANA U]*[FANA G]*[FANA |
| | | C]*[FANA C] |
| SEQ ID NO: 23 | AUM-FANA-FXP3-3 | [FANA A]*[FANA A]*[FANA U]*[FANA A]*[FANA |
| | | U]*[FANA U]*A*G*G*A*T*G*G*T*G*[FANA |
| | | U]*[FANA C]*[FANA U]*[FANA G]*[FANA |
| | | U]*[FANA C] |
| SEQ ID NO: 24 | AUM-FANA-FXP3-4 | [FANA G]*[FANA A]*[FANA A]*[FANA G]*[FANA |
| | | U]*[FANA U]*G*C*C*G*G*G*A*G*A*[FANA |
| | | G]*[FANA C]*[FANA U]*[FANA G]*[FANA |
| | | A]*[FANA A] |
| SEQ ID NO: 25 | AUM-FANA-FXP3-5; | [FANA G]*[FANA G]*[FANA U]*[FANA U]*[FANA |
| | AUM-FANA-5 | G]*[FANA C]*T*G*T*C*T*T*T*C*C*[FANA |
| | | U]*[FANA G]*[FANA G]*[FANA G]*[FANA |
| | | U]*[FANA G] |
| SEQ ID NO: 26 | AUM-FANA-FXP3-6; | [FANA A]*[FANA G]*[FANA U]*[FANA U]*[FANA |
| | AUM-FANA-6 | G]*[FANA C]*T*G*C*T*T*T*A*G*G*[FANA |
| | | U]*[FANA G]*[FANA G]*[FANA A]*[FANA |
| | | G]*[FANA U] |
| SEQ ID NO: 27 | AUM-FANA-FXP3-7 | [FANA G]*[FANA A]*[FANA U]*[FANA G]*[FANA |
| | | U]*[FANA G]*A*C*T*G*T*C*T*T*C*[FANA |
| | | C]*[FANA A]*[FANA A]*[FANA G]*[FANA |
| | | U]*[FANA C] |
| SEQ ID NO: 28 | AUM-FANA-FXP3-8 | [FANA U]*[FANA G]*[FANA A]*[FANA A]*[FANA |
| | | U]*[FANA U]*C*A*G*T*A*C*T*G*C*[FANA |
| | | A]*[FANA G]*[FANA A]*[FANA G]*[FANA |
| | | C]*[FANA U] |
| SEQ ID NO: 29 | AUM-FANA-FXP3-9 | [FANA C]*[FANA U]*[FANA U]*[FANA U]*[FANA |
| | | G]*[FANA A]*C*T*G*G*A*A*A*G*T*[FANA |
| | | U]*[FANA C]*[FANA A]*[FANA U]*[FANA |
| | | C]*[FANA U] |
| SEQ ID NO: 30 | Scramble Control | [FANA U]*[FANA G]*[FANA A]*[FANA C]*[FANA |
| | | C] [FANA C]*[FANA U]*A*T*G*C*T*G*T*[FANA |
| | | U]*[FANA C]*[FANA C]*[FANA U]*[FANA |
| | | A]*[FANA U]*[FANA A] |
| SEQ ID NO: 31 | AUM-F-FXP3-11 | AGAUGAAGCCTTGGTCAGUGC |
| SEQ ID NO: 32 | AUM-F-FXP3-12 | UUUCUTGCGGAACTCCAGCUC |
| SEQ ID NO: 33 | AUM-F-FXP3-13 | ACAGGTGAATTCTAACAGGCC |
| SEQ ID NO: 34 | AUM-F-FXP3-14 | UAGUUCCTCTGCAGTCUAAGC |
| SEQ ID NO: 35 | AUM-F-FXP3-15 | AUAAATGAGTAGTTCCUCUGC |
| SEQ ID NO: 36 | AUM-F-FXP3-16 | UUUGAGTGTACTGAGGCAGGC |
| SEQ ID NO: 37 | AUM-F-FXP3-17 | GUUCAAGGAAGAAGAGGAGGC |
| SEQ ID NO: 38 | AUM-F-FXP3-18 | ACUGACCAAGGCTTCAUCUGU |
| SEQ ID NO: 39 | AUM-F-FXP3-19 | AAACAGCACATTCCCAGAGUU |
| SEQ ID NO: 40 | AUM-F-FXP3-20 | UUGUGAAGGCTCTGTTUGGCU |
| SEQ ID NO: 41 | AUM-F-FXP3-21 | GUUGUGAAGGCTCTGTUUGGC |
| SEQ ID NO: 42 | AUM-F-FXP3-22 | GUUGUTTGAGTGTACTGAGGC |
| SEQ ID NO: 43 | AUM-F-FXP3-23 | AGGUGAATTCTAACAGGCCGU |
| SEQ ID NO: 44 | AUM-F-FXP3-24 | GUCUAAGCTGAGGCATGGAUC |
| SEQ ID NO: 45 | AUM-F-FXP3-25 | AGUCUAAGCTGAGGCAUGGAU |
| SEQ ID NO: 46 | AUM-F-FXP3-26 | GAUGATGCCACAGATGAAGCC |
| SEQ ID NO: 47 | AUM-F-FXP3-27 | UGUUGAGTGAGGGACAGGAUU |
| SEQ ID NO: 48 | AUM-F-FXP3-28 | AACCUCAAAGCTGCATCAUCA |
| SEQ ID NO: 49 | AUM-F-FXP3-29 | CACAGGTGAATTCTAACAGGC |
| SEQ ID NO: 50 | AUM-F-FXP3-30 | AGUCUAAGCTGAGGCAUGGAU |
| SEQ ID NO: 51 | AUM-F-FXP3-31 | AUGUACCAAGGCAGGCUCUUC |
| SEQ ID NO: 52 | AUM-F-FXP3-32 | AAUAUTAGGATGGTGTCUGUC |
| SEQ ID NO: 53 | AUM-F-FXP3-33 | UAUAGCTGGTTGTGAGGGCUC |
| SEQ ID NO: 54 | AUM-F-FXP3-34 | UCAUUGAGTGTCCTCTGCCUC |
| SEQ ID NO: 55 | AUM-F-FXP3-35 | ACUAACAGCTAGAGGCUUUGC |
| SEQ ID NO: 56 | AUM-F-FXP3-36 | CCACUTGCAGACTCCAUUUGC |
| SEQ ID NO: 57 | AUM-F-FXP3-37 | AUAGGTACACGTAGGACUUGC |
| SEQ ID NO: 58 | AUM-F-FXP3-38 | AUUUCATTGAGTGTCCUCUGC |
| SEQ ID NO: 59 | AUM-F-FXP3-39 | UAGAUTGAGACAGAGAUGGGC |
| SEQ ID NO: 60 | AUM-F-FXP3-40 | UGACUGGATGTAAGTAUAGGC |
| SEQ ID NO: 61 | AUM-F-FXP3-41 | UGUUCAAGGTATGGAGCAGGC |
| SEQ ID NO: 62 | AUM-F-FXP3-42 | AGUUCACGAATGTACCAAGGC |
| SEQ ID NO: 63 | AUM-F-FXP3-43 | UGUUACGGGAATAGGAGGAGC |
| SEQ ID NO: 64 | AUM-F-FXP3-44 | CUAACAGCTAGAGGCTUUGCC |
| SEQ ID NO: 65 | AUM-F-FXP3-45 | UAGGUACACGTAGGACUUGCC |
| SEQ ID NO: 66 | AUM-F-FXP3-46 | UUUCATTGAGTGTCCTCUGCC |
| SEQ ID NO: 67 | AUM-F-FXP3-47 | AUAGUTACCCTGGTTTCUACC |
| SEQ ID NO: 68 | AUM-F-FXP3-48 | UUCUACCAGATCTTGTCGGAC |
| SEQ ID NO: 69 | AUM-F-FXP3-49 | UUCUAGCACTCTCTTTCAUUU |
| SEQ ID NO: 70 | AUM-F-FXP3-50 | AAGUATAGGCACGTGAGUGUU |
| SEQ ID NO: 71 | AUM-F-FXP3-51 | UCACUACTAGGCAGAGCUGUU |
| SEQ ID NO: 72 | AUM-F-FXP3-52 | ACAACTAACAGCTAGAGGCUU |
| SEQ ID NO: 73 | AUM-F-FXP3-53 | ACACUCACGTGCCTATACUUA |
| SEQ ID NO: 74 | AUM-F-FXP3-54 | UUCAUTTGGTATCCGCUUUCU |
| SEQ ID NO: 75 | AUM-F-FXP3-55 | AGUGAGACGTGGGGTACAUCU |
| SEQ ID NO: 76 | AUM-F-FXP3-56 | UGGUATCCGCTTTCTCCUGCU |
| SEQ ID NO: 77 | AUM-F-FXP3-57 | UUCAUTGAGTGTCCTCUGCCU |
| SEQ ID NO: 78 | AUM-F-FXP3-58 | ACUAGTAAAGGGTGGTCUUAU |
| SEQ ID NO: 79 | AUM-F-FXP3-59 | AUAGUGCCCGTGGTTCCAGAU |
| SEQ ID NO: 80 | AUM-F-FXP3-60 | UGUUCTAGCACTCTCTUUCAU |
| SEQ ID NO: 81 | AUM-F-FXP3-61 | UAUUAGGATGGTGTCTGUCAU |
| SEQ ID NO: 82 | AUM-F-FXP3-62 | CAAUACCTCTCTGCCACUUUC |
| SEQ ID NO: 83 | AUM-F-FXP3-63 | CUAAGAAGGATGATGCUGUUC |
| SEQ ID NO: 84 | AUM-F-FXP3-64 | CACUACTAGGCAGAGCUGUUC |
| SEQ ID NO: 85 | AUM-F-FXP3-65 | AGAUGAAGCCTTGGTCAGUGC |
| SEQ ID NO: 86 | AUM-F-FXP3-66 | UUUCUUGCGGAACTCCAGCUC |
| SEQ ID NO: 87 | AUM-F-FXP3-67 | ACAGGUGAATTCTAACAGGCC |
| SEQ ID NO: 88 | AUM-F-FXP3-68 | UAGUUCCTCTGCAGTCUAAGC |
| SEQ ID NO: 89 | AUM-F-FXP3-69 | AUAAAUGAGTAGTTCCUCUGC |
| SEQ ID NO: 90 | AUM-F-FXP3-70 | UUUGAGTGTACTGAGGCAGGC |
| SEQ ID NO: 91 | AUM-F-FXP3-71 | GUUCAAGGAAGAAGAGGAGGC |
| SEQ ID NO: 92 | AUM-F-FXP3-72 | ACUGACCAAGGCTTCAUCUGU |
| SEQ ID NO: 93 | AUM-F-FXP3-73 | AAACAGCACATTCCCAGAGUU |
| SEQ ID NO: 94 | AUM-F-FXP3-74 | UUGUGAAGGCTCTGTUUGGCU |
| SEQ ID NO: 95 | AUM-F-FXP3-75 | GUUGUGAAGGCTCTGUUUGGC |
| SEQ ID NO: 96 | AUM-F-FXP3-76 | GUUGUUTGAGTGTACUGAGGC |
| SEQ ID NO: 97 | AUM-F-FXP3-77 | AGGUGAATTCTAACAGGCCGU |
| SEQ ID NO: 98 | AUM-F-FXP3-78 | GUCUAAGCTGAGGCAUGGAUC |
| SEQ ID NO: 99 | AUM-F-FXP3-79 | AGUCUAAGCTGAGGCAUGGAU |
| SEQ ID NO: 100 | AUM-F-FXP3-80 | GAUGAUGCCACAGATGAAGCC |
| SEQ ID NO: 101 | AUM-F-FXP3-81 | UGUUGAGTGAGGGACAGGAUU |
| SEQ ID NO: 102 | AUM-F-FXP3-82 | AACCUCAAAGCTGCATCAUCA |
| SEQ ID NO: 103 | AUM-F-FXP3-83 | CACAGGTGAATTCTAACAGGC |
| SEQ ID NO: 104 | AUM-F-FXP3-84 | AGUCUAAGCTGAGGCAUGGAU |
| SEQ ID NO: 105 | AUM-F-FXP3-85 | AUGUACCAAGGCAGGCUCUUC |
| SEQ ID NO: 106 | AUM-F-FXP3-86 | AAUAUUAGGATGGTGUCUGUC |
| SEQ ID NO: 107 | AUM-F-FXP3-87 | UAUAGCTGGTTGTGAGGGCUC |
| SEQ ID NO: 108 | AUM-F-FXP3-88 | UCAUUGAGTGTCCTCUGCCUC |
| SEQ ID NO: 109 | AUM-F-FXP3-89 | ACUAACAGCTAGAGGCUUUGC |
| SEQ ID NO: 110 | AUM-F-FXP3-90 | CCACUUGCAGACTCCAUUUGC |
| SEQ ID NO: 111 | AUM-F-FXP3-91 | AUAGGUACACGTAGGACUUGC |
| SEQ ID NO: 112 | AUM-F-FXP3-92 | AUUUCATTGAGTGTCCUCUGC |
| SEQ ID NO: 113 | AUM-F-FXP3-93 | UAGAUUGAGACAGAGAUGGGC |
| SEQ ID NO: 114 | AUM-F-FXP3-94 | UGACUGGATGTAAGTAUAGGC |
| SEQ ID NO: 115 | AUM-F-FXP3-95 | UGUUCAAGGTATGGAGCAGGC |
| SEQ ID NO: 116 | AUM-F-FXP3-96 | AGUUCACGAATGTACCAAGGC |
| SEQ ID NO: 117 | AUM-F-FXP3-97 | UGUUACGGGAATAGGAGGAGC |
| SEQ ID NO: 118 | AUM-F-FXP3-98 | CUAACAGCTAGAGGCUUUGCC |
| SEQ ID NO: 119 | AUM-F-FXP3-99 | UAGGUACACGTAGGACUUGCC |
| SEQ ID NO: 120 | AUM-F-FXP3-100 | UUUCAUTGAGTGTCCUCUGCC |
| SEQ ID NO: 121 | AUM-F-FXP3-101 | AUAGUUACCCTGGTTUCUACC |
| SEQ ID NO: 122 | AUM-F-FXP3-102 | UUCUACCAGATCTTGUCGGAC |
| SEQ ID NO: 123 | AUM-F-FXP3-103 | UUCUAGCACTCTCTTUCAUUU |
| SEQ ID NO: 124 | AUM-F-FXP3-104 | AAGUAUAGGCACGTGAGUGUU |
| SEQ ID NO: 125 | AUM-F-FXP3-105 | UCACUACTAGGCAGAGCUGUU |
| SEQ ID NO: 126 | AUM-F-FXP3-106 | ACAACUAACAGCTAGAGGCUU |
| SEQ ID NO: 127 | AUM-F-FXP3-107 | ACACUCACGTGCCTAUACUUA |
| SEQ ID NO: 128 | AUM-F-FXP3-108 | UUCAUUTGGTATCCGCUUUCU |
| SEQ ID NO: 129 | AUM-F-FXP3-109 | AGUGAGACGTGGGGTACAUCU |
| SEQ ID NO: 130 | AUM-F-FXP3-110 | UGGUAUCCGCTTTCTCCUGCU |
| SEQ ID NO: 131 | AUM-F-FXP3-111 | UUCAUUGAGTGTCCTCUGCCU |
| SEQ ID NO: 132 | AUM-F-FXP3-112 | ACUAGUAAAGGGTGGUCUUAU |
| SEQ ID NO: 133 | AUM-F-FXP3-113 | AUAGUGCCCGTGGTTCCAGAU |
| SEQ ID NO: 134 | AUM-F-FXP3-114 | UGUUCUAGCACTCTCUUUCAU |
| SEQ ID NO: 135 | AUM-F-FXP3-115 | UAUUAGGATGGTGTCUGUCAU |
| SEQ ID NO: 136 | AUM-F-FXP3-116 | CAAUACCTCTCTGCCACUUUC |
| SEQ ID NO: 137 | AUM-F-FXP3-117 | CUAAGAAGGATGATGCUGUUC |
| SEQ ID NO: 138 | AUM-F-FXP3-118 | CACUACTAGGCAGAGCUGUUC |
| SEQ ID NO: 139 | AUM-FXP3-11 | AGATGAAGCCTTGGTCAGTGC |
| SEQ ID NO: 140 | AUM-FXP3-12 | TTTCTTGCGGAACTCCAGCTC |
| SEQ ID NO: 141 | AUM-FXP3-13 | ACAGGTGAATTCTAACAGGCC |
| SEQ ID NO: 142 | AUM-FXP3-14 | TAGTTCCTCTGCAGTCTAAGC |
| SEQ ID NO: 143 | AUM-FXP3-15 | ATAAATGAGTAGTTCCTCTGC |
| SEQ ID NO: 144 | AUM-FXP3-16 | TTTGAGTGTACTGAGGCAGGC |
| SEQ ID NO: 145 | AUM-FXP3-17 | GTTCAAGGAAGAAGAGGAGGC |
| SEQ ID NO: 146 | AUM-FXP3-18 | ACTGACCAAGGCTTCATCTGT |
| SEQ ID NO: 147 | AUM-FXP3-19 | AAACAGCACATTCCCAGAGTT |
| SEQ ID NO: 148 | AUM-FXP3-20 | TTGTGAAGGCTCTGTTTGGCT |
| SEQ ID NO: 149 | AUM-FXP3-21 | GTTGTGAAGGCTCTGTTTGGC |
| SEQ ID NO: 150 | AUM-FXP3-22 | GTTGTTTGAGTGTACTGAGGC |
| SEQ ID NO: 151 | AUM-FXP3-23 | AGGTGAATTCTAACAGGCCGT |
| SEQ ID NO: 152 | AUM-FXP3-24 | GTCTAAGCTGAGGCATGGATC |
| SEQ ID NO: 153 | AUM-FXP3-25 | AGTCTAAGCTGAGGCATGGAT |
| SEQ ID NO: 154 | AUM-FXP3-26 | GATGATGCCACAGATGAAGCC |
| SEQ ID NO: 155 | AUM-FXP3-27 | TGTTGAGTGAGGGACAGGATT |
| SEQ ID NO: 156 | AUM-FXP3-28 | AACCTCAAAGCTGCATCATCA |
| SEQ ID NO: 157 | AUM-FXP3-29 | CACAGGTGAATTCTAACAGGC |
| SEQ ID NO: 158 | AUM-FXP3-30 | AGTCTAAGCTGAGGCATGGAT |
| SEQ ID NO: 159 | AUM-FXP3-31 | ATGTACCAAGGCAGGCTCTTC |
| SEQ ID NO: 160 | AUM-FXP3-32 | AATATTAGGATGGTGTCTGTC |
| SEQ ID NO: 161 | AUM-FXP3-33 | TATAGCTGGTTGTGAGGGCTC |
| SEQ ID NO: 162 | AUM-FXP3-34 | TCATTGAGTGTCCTCTGCCTC |
| SEQ ID NO: 163 | AUM-FXP3-35 | ACTAACAGCTAGAGGCTTTGC |
| SEQ ID NO: 164 | AUM-FXP3-36 | CCACTTGCAGACTCCATTTGC |
| SEQ ID NO: 165 | AUM-FXP3-37 | ATAGGTACACGTAGGACTTGC |
| SEQ ID NO: 166 | AUM-FXP3-38 | ATTTCATTGAGTGTCCTCTGC |
| SEQ ID NO: 167 | AUM-FXP3-39 | TAGATTGAGACAGAGATGGGC |
| SEQ ID NO: 168 | AUM-FXP3-40 | TGACTGGATGTAAGTATAGGC |
| SEQ ID NO: 169 | AUM-FXP3-41 | TGTTCAAGGTATGGAGCAGGC |
| SEQ ID NO: 170 | AUM-FXP3-42 | AGTTCACGAATGTACCAAGGC |
| SEQ ID NO: 171 | AUM-FXP3-43 | TGTTACGGGAATAGGAGGAGC |
| SEQ ID NO: 172 | AUM-FXP3-44 | CTAACAGCTAGAGGCTTTGCC |
| SEQ ID NO: 173 | AUM-FXP3-45 | TAGGTACACGTAGGACTTGCC |
| SEQ ID NO: 174 | AUM-FXP3-46 | TTTCATTGAGTGTCCTCTGCC |
| SEQ ID NO: 175 | AUM-FXP3-47 | ATAGTTACCCTGGTTTCTACC |
| SEQ ID NO: 176 | AUM-FXP3-48 | TTCTACCAGATCTTGTCGGAC |
| SEQ ID NO: 177 | AUM-FXP3-49 | TTCTAGCACTCTCTTTCATTT |
| SEQ ID NO: 178 | AUM-FXP3-50 | AAGTATAGGCACGTGAGTGTT |
| SEQ ID NO: 179 | AUM-FXP3-51 | TCACTACTAGGCAGAGCTGTT |
| SEQ ID NO: 180 | AUM-FXP3-52 | ACAACTAACAGCTAGAGGCTT |
| SEQ ID NO: 181 | AUM-FXP3-53 | ACACTCACGTGCCTATACTTA |
| SEQ ID NO: 182 | AUM-FXP3-54 | TTCATTTGGTATCCGCTTTCT |
| SEQ ID NO: 183 | AUM-FXP3-55 | AGTGAGACGTGGGGTACATCT |
| SEQ ID NO: 184 | AUM-FXP3-56 | TGGTATCCGCTTTCTCCTGCT |
| SEQ ID NO: 185 | AUM-FXP3-57 | TTCATTGAGTGTCCTCTGCCT |
| SEQ ID NO: 186 | AUM-FXP3-58 | ACTAGTAAAGGGTGGTCTTAT |
| SEQ ID NO: 187 | AUM-FXP3-59 | ATAGTGCCCGTGGTTCCAGAT |
| SEQ ID NO: 188 | AUM-FXP3-60 | TGTTCTAGCACTCTCTTTCAT |
| SEQ ID NO: 189 | AUM-FXP3-61 | TATTAGGATGGTGTCTGTCAT |
| SEQ ID NO: 190 | AUM-FXP3-62 | CAATACCTCTCTGCCACTTTC |
| SEQ ID NO: 191 | AUM-FXP3-63 | CTAAGAAGGATGATGCTGTTC |
| SEQ ID NO: 192 | AUM-FXP3-64 | CACTACTAGGCAGAGCTGTTC |
| SEQ ID NO: 193 | AUM-FANA-FXP3-10 | [FANA A]*[FANA G]*[FANA U]*[FANA U]*[FANA |
| | | G]*C*T*G*C*T*T*T*A*G*G*T*[FANA G]*[FANA |
| | | G]*[FANA A]*[FANA G]*[FANA U] |
| SEQ ID NO: 194 | AUM-FANA-FXP3-11 | [FANA A]*[FANA G]*[FANA A]*[FANA U]*[FANA |
| | | G]*A*A*G*C*C*T*T*G*G*T*C*[FANA A]*[FANA |
| | | G]*[FANA U]*[FANA G]*[FANA C] |
| SEQ ID NO: 195 | AUM-FANA-FXP3-12 | [FANA U]*[FANA U]*[FANA U]*[FANA C]*[FANA |
| | | U]*T*G*C*G*G*A*A*C*T*C*C*[FANA A]*[FANA |
| | | G]*[FANA C]*[FANA U]*[FANA C] |
| SEQ ID NO: 196 | AUM-FANA-FXP3-13 | [FANA A]*[FANA C]*[FANA A]*[FANA G]*[FANA |
| | | G]*T*G*A*A*T*T*C*T*A*A*C*[FANA A]*[FANA |
| | | G]*[FANA G]*[FANA C]*[FANA C] |
| SEQ ID NO: 197 | AUM-FANA-FXP3-14 | [FANA U]*[FANA A]*[FANA G]*[FANA U]*[FANA |
| | | U]*C*C*T*C*T*G*C*A*G*T*C*[FANA U]*[FANA |
| | | A]*[FANA A]*[FANA G]*[FANA C] |
| SEQ ID NO: 198 | AUM-FANA-FXP3-15 | [FANA A]*[FANA U]*[FANA A]*[FANA A]*[FANA |
| | | A]*T*G*A*G*T*A*G*T*T*C*C*[FANA U]*[FANA |
| | | C]*[FANA U]*[FANA G]*[FANA C] |
| SEQ ID NO: 199 | AUM-FANA-FXP3-16 | [FANA U]*[FANA U]*[FANA U]*[FANA G]*[FANA |
| | | A]*G*T*G*T*A*C*T*G*A*G*G*[FANA C]*[FANA |
| | | A]*[FANA G]*[FANA G]*[FANA C] |
| SEQ ID NO: 200 | AUM-FANA-FXP3-17 | [FANA G]*[FANA U]*[FANA U]*[FANA C]*[FANA |
| | | A]*A*G*G*A*A*G*A*A*G*A*G*[FANA G]*[FANA |
| | | A]*[FANA G]*[FANA G]*[FANA C] |
| SEQ ID NO: 201 | AUM-FANA-FXP3-18 | [FANA A]*[FANA C]*[FANA U]*[FANA G]*[FANA |
| | | A]*C*C*A*A*G*G*C*T*T*C*A*[FANA U]*[FANA |
| | | C]*[FANA U]*[FANA G]*[FANA U] |
| SEQ ID NO: 202 | AUM-FANA-FXP3-19 | [FANA A]*[FANA A]*[FANA A]*[FANA C]*[FANA |
| | | A]*G*C*A*C*A*T*T*C*C*C*A*[FANA G]*[FANA |
| | | A]*[FANA G]*[FANA U]*[FANA U] |
| SEQ ID NO: 203 | AUM-FANA-FXP3-20 | [FANA U]*[FANA U]*[FANA G]*[FANA U]*[FANA |
| | | G]*A*A*G*G*C*T*C*T*G*T*T*[FANA U]*[FANA |
| | | G]*[FANA G]*[FANA C]*[FANA U] |
| SEQ ID NO: 204 | AUM-FANA-FXP3-21 | [FANA G]*[FANA U]*[FANA U]*[FANA G]*[FANA |
| | | U]*G*A*A*G*G*C*T*C*T*G*T*[FANA U]*[FANA |
| | | U]*[FANA G]*[FANA G]*[FANA C] |
| SEQ ID NO: 205 | AUM-FANA-FXP3-22 | [FANA G]*[FANA U]*[FANA U]*[FANA G]*[FANA |
| | | U]*T*T*G*A*G*T*G*T*A*C*T*[FANA G]*[FANA |
| | | A]*[FANA G]*[FANA G]*[FANA C] |
| SEQ ID NO: 206 | AUM-FANA-FXP3-23 | [FANA A]*[FANA G]*[FANA G]*[FANA U]*[FANA |
| | | G]*A*A*T*T*C*T*A*A*C*A*G*[FANA G]*[FANA |
| | | C]*[FANA C]*[FANA G]*[FANA U] |
| SEQ ID NO: 207 | AUM-FANA-FXP3-24 | [FANA G]*[FANA U]*[FANA C]*[FANA U]*[FANA |
| | | A]*A*G*C*T*G*A*G*G*C*A*T*[FANA G]*[FANA |
| | | G]*[FANA A]*[FANA U]*[FANA C] |
| SEQ ID NO: 208 | AUM-FANA-FXP3-25 | [FANA A]*[FANA G]*[FANA U]*[FANA C]*[FANA |
| | | U]*A*A*G*C*T*G*A*G*G*C*A*[FANA U]*[FANA |
| | | G]*[FANA G]*[FANA A]*[FANA U] |
| SEQ ID NO: 209 | AUM-FANA-FXP3-26 | [FANA G]*[FANA A]*[FANA U]*[FANA G]*[FANA |
| | | A]*T*G*C*C*A*C*A*G*A*T*G*[FANA A]*[FANA |
| | | A]*[FANA G]*[FANA C]*[FANA C] |
| SEQ ID NO: 210 | AUM-FANA-FXP3-27 | [FANA U]*[FANA G]*[FANA U]*[FANA U]*[FANA |
| | | G]*A*G*T*G*A*G*G*G*A*C*A*[FANA G]*[FANA |
| | | G]*[FANA A]*[FANA U]*[FANA U] |
| SEQ ID NO: 211 | AUM-FANA-FXP3-28 | [FANA A]*[FANA A]*[FANA C]*[FANA C]*[FANA |
| | | U]*C*A*A*A*G*C*T*G*C*A*T*C*[FANA A]*[FANA |
| | | U]*[FANA C]*[FANA A]*[FANA ] |
| SEQ ID NO: 212 | AUM-FANA-FXP3-29 | [FANA C]*[FANA A]*[FANA C]*[FANA A]*[FANA |
| | | G]*G*T*G*A*A*T*T*C*T*A*A*[FANA C]*[FANA |
| | | A]*[FANA G]*[FANA G]*[FANA C] |
| SEQ ID NO: 213 | AUM-FANA-FXP3-30 | [FANA A]*[FANA G]*[FANA U]*[FANA C]*[FANA |
| | | U]*A*A*G*C*T*G*A*G*G*C*A*[FANA U]*[FANA |
| | | G]*[FANA G]*[FANA A]*[FANA U] |
| SEQ ID NO: 214 | AUM-FANA-FXP3-31 | [FANA A]*[FANA U]*[FANA G]*[FANA U]*[FANA |
| | | A]*C*C*A*A*G*G*C*A*G*G*C*[FANA U]*[FANA |
| | | C]*[FANA U]*[FANA U]*[FANA C] |
| SEQ ID NO: 215 | AUM-FANA-FXP3-32 | [FANA A]*[FANA A]*[FANA U]*[FANA A]*[FANA |
| | | U]*T*A*G*G*A*T*G*G*T*G*T*[FANA C]*[FANA |
| | | U]*[FANA G]*[FANA U]*[FANA C] |
| SEQ ID NO: 216 | AUM-FANA-FXP3-33 | [FANA U]*[FANA A]*[FANA U]*[FANA A]*[FANA |
| | | G]*C*T*G*G*T*T*G*T*G*A*G*[FANA G]*[FANA |
| | | G]*[FANA C]*[FANA U]*[FANA C] |
| SEQ ID NO: 217 | AUM-FANA-FXP3-34 | [FANA U]*[FANA C]*[FANA A]*[FANA U]*[FANA |
| | | U]*G*A*G*T*G*T*C*C*T*C*T*[FANA G]*[FANA |
| | | C]*[FANA C]*[FANA U]*[FANA C] |
| SEQ ID NO: 218 | AUM-FANA-FXP3-35 | [FANA A]*[FANA C]*[FANA U]*[FANA A]*[FANA |
| | | A]*C*A*G*C*T*A*G*A*G*G*C*[FANA U]*[FANA |
| | | U]*[FANA U]*[FANA G]*[FANA C] |
| SEQ ID NO: 219 | AUM-FANA-FXP3-36 | [FANA C]*[FANA C]*[FANA A]*[FANA C]*[FANA |
| | | U]*T*G*C*A*G*A*C*T*C*C*A*[FANA U]*[FANA |
| | | U]*[FANA U]*[FANA G]*[FANA C] |
| SEQ ID NO: 220 | AUM-FANA-FXP3-37 | [FANA A]*[FANA U]*[FANA A]*[FANA G]*[FANA |
| | | G]*T*A*C*A*C*G*T*A*G*G*A*[FANA C]*[FANA |
| | | U]*[FANA U]*[FANA G]*[FANA C] |
| SEQ ID NO: 221 | AUM-FANA-FXP3-38 | [FANA A]*[FANA U]*[FANA U]*[FANA U]*[FANA |
| | | C]*A*T*T*G*A*G*T*G*T*C*C*[FANA U]*[FANA |
| | | C]*[FANA U]*[FANA G]*[FANA C] |
| SEQ ID NO: 222 | AUM-FANA-FXP3-39 | [FANA U]*[FANA A]*[FANA G]*[FANA A]*[FANA |
| | | U]*T*G*A*G*A*C*A*G*A*G*A*[FANA U]*[FANA |
| | | G]*[FANA G]*[FANA G]*[FANA C] |
| SEQ ID NO: 223 | AUM-FANA-FXP3-40 | [FANA U]*[FANA G]*[FANA A]*[FANA C]*[FANA |
| | | U]*G*G*A*T*G*T*A*A*G*T*A*[FANA U]*[FANA |
| | | A]*[FANA G]*[FANA G]*[FANA C] |
| SEQ ID NO: 224 | AUM-FANA-FXP3-41 | [FANA U]*[FANA G]*[FANA U]*[FANA U]*[FANA |
| | | C]*A*A*G*G*T*A*T*G*G*A*G*[FANA C]*[FANA |
| | | A]*[FANA G]*[FANA G]*[FANA C] |
| SEQ ID NO: 225 | AUM-FANA-FXP3-42 | [FANA A]*[FANA G]*[FANA U]*[FANA U]*[FANA |
| | | C]*A*C*G*A*A*T*G*T*A*C*C*[FANA A]*[FANA |
| | | A]*[FANA G]*[FANA G]*[FANA C] |
| SEQ ID NO: 226 | AUM-FANA-FXP3-43 | [FANA U]*[FANA G]*[FANA U]*[FANA U]*[FANA |
| | | A]*C*G*G*G*A*A*T*A*G*G*A*[FANA G]*[FANA |
| | | G]*[FANA A]*[FANA G]*[FANA C] |
| SEQ ID NO: 227 | AUM-FANA-FXP3-44 | [FANA C]*[FANA U]*[FANA A]*[FANA A]*[FANA |
| | | C]*A*G*C*T*A*G*A*G*G*C*T*[FANA U]*[FANA |
| | | U]*[FANA G]*[FANA C]*[FANA C] |
| SEQ ID NO: 228 | AUM-FANA-FXP3-45 | [FANA U]*[FANA A]*[FANA G]*[FANA G]*[FANA |
| | | U]*A*C*A*C*G*T*A*G*G*A*C*[FANA U]*[FANA |
| | | U]*[FANA G]*[FANA C]*[FANA C] |
| SEQ ID NO: 229 | AUM-FANA-FXP3-46 | [FANA U]*[FANA U]*[FANA U]*[FANA C]*[FANA |
| | | A]*T*T*G*A*G*T*G*T*C*C*T*[FANA C]*[FANA |
| | | U]*[FANA G]*[FANA C]*[FANA C] |
| SEQ ID NO: 230 | AUM-FANA-FXP3-47 | [FANA A]*[FANA U]*[FANA A]*[FANA G]*[FANA |
| | | U]*T*A*C*C*C*T*G*G*T*T*T*[FANA C]*[FANA |
| | | U]*[FANA A]*[FANA C]*[FANA C] |
| SEQ ID NO: 231 | AUM-FANA-FXP3-48 | [FANA U]*[FANA U]*[FANA C]*[FANA U]*[FANA |
| | | A]*C*C*A*G*A*T*C*T*T*G*T*[FANA C]*[FANA |
| | | G]*[FANA G]*[FANA A]*[FANA C] |
| SEQ ID NO: 232 | AUM-FANA-FXP3-49 | [FANA U]*[FANA U]*[FANA C]*[FANA U]*[FANA |
| | | A]*G*C*A*C*T*C*T*C*T*T*T*[FANA C]*[FANA |
| | | A]*[FANA U]*[FANA U]*[FANA U] |
| SEQ ID NO: 233 | AUM-FANA-FXP3-50 | [FANA A]*[FANA A]*[FANA G]*[FANA U]*[FANA |
| | | A]*T*A*G*G*C*A*C*G*T*G*A*[FANA G]*[FANA |
| | | U]*[FANA G]*[FANA U]*[FANA U] |
| SEQ ID NO: 234 | AUM-FANA-FXP3-51 | [FANA U]*[FANA C]*[FANA A]*[FANA C]*[FANA |
| | | U]*A*C*T*A*G*G*C*A*G*A*G*[FANA C]*[FANA |
| | | U]*[FANA G]*[FANA U]*[FANA U] |
| SEQ ID NO: 235 | AUM-FANA-FXP3-52 | [FANA A]*[FANA C]*[FANA A]*[FANA A]*[FANA |
| | | C]*T*A*A*C*A*G*C*T*A*G*A*[FANA G]*[FANA |
| | | G]*[FANA C]*[FANA U]*[FANA U] |
| SEQ ID NO: 236 | AUM-FANA-FXP3-53 | [FANA A]*[FANA C]*[FANA A]*[FANA C]*[FANA |
| | | U]*C*A*C*G*T*G*C*C*T*A*T*[FANA A]*[FANA |
| | | C]*[FANA U]*[FANA U]*[FANA A] |
| SEQ ID NO: 237 | AUM-FANA-FXP3-54 | [FANA U]*[FANA U]*[FANA C]*[FANA A]*[FANA |
| | | U]*T*T*G*G*T*A*T*C*C*G*C*[FANA U]*[FANA |
| | | U]*[FANA U]*[FANA C]*[FANA U] |
| SEQ ID NO: 238 | AUM-FANA-FXP3-55 | [FANA A]*[FANA G]*[FANA U]*[FANA G]*[FANA |
| | | A]*G*A*C*G*T*G*G*G*G*T*A*[FANA C]*[FANA |
| | | A]*[FANA U]*[FANA C]*[FANA U] |
| SEQ ID NO: 239 | AUM-FANA-FXP3-56 | [FANA U]*[FANA G]*[FANA G]*[FANA U]*[FANA |
| | | A]*T*C*C*G*C*T*T*T*C*T*C*[FANA C]*[FANA |
| | | U]*[FANA G]*[FANA C]*[FANA U] |
| SEQ ID NO: 240 | AUM-FANA-FXP3-57 | [FANA U]*[FANA U]*[FANA C]*[FANA A]*[FANA |
| | | U]*T*G*A*G*T*G*T*C*C*T*C*[FANA U]*[FANA |
| | | G]*[FANA C]*[FANA C]*[FANA U] |
| SEQ ID NO: 241 | AUM-FANA-FXP3-58 | [FANA A]*[FANA C]*[FANA U]*[FANA A]*[FANA |
| | | G]*T*A*A*A*G*G*G*T*G*G*T*[FANA C]*[FANA |
| | | U]*[FANA U]*[FANA A]*[FANA U] |
| SEQ ID NO: 242 | AUM-FANA-FXP3-59 | [FANA A]*[FANA U]*[FANA A]*[FANA G]*[FANA |
| | | U]*G*C*C*C*G*T*G*G*T*T*C*[FANA C]*[FANA |
| | | A]*[FANA G]*[FANA A]*[FANA U] |
| SEQ ID NO: 243 | AUM-FANA-FXP3-60 | [FANA U]*[FANA G]*[FANA U]*[FANA U]*[FANA |
| | | C]*T*A*G*C*A*C*T*C*T*C*T*[FANA U]*[FANA |
| | | U]*[FANA C]*[FANA A]*[FANA U] |
| SEQ ID NO: 244 | AUM-FANA-FXP3-61 | [FANA U]*[FANA A]*[FANA U]*[FANA U]*[FANA |
| | | A]*G*G*A*T*G*G*T*G*T*C*T*[FANA G]*[FANA |
| | | U]*[FANA C]*[FANA A]*[FANA U] |
| SEQ ID NO: 245 | AUM-FANA-FXP3-62 | [FANA C]*[FANA A]*[FANA A]*[FANA U]*[FANA |
| | | A]*C*C*T*C*T*C*T*G*C*C*A*[FANA C]*[FANA |
| | | U]*[FANA U]*[FANA U]*[FANA C] |
| SEQ ID NO: 246 | AUM-FANA-FXP3-63 | [FANA C]*[FANA U]*[FANA A]*[FANA A]*[FANA |
| | | G]*A*A*G*G*A*T*G*A*T*G*C*[FANA U]*[FANA |
| | | G]*[FANA U]*[FANA U]*[FANA C] |
| SEQ ID NO: 247 | AUM-FANA-FXP3-64 | [FANA C]*[FANA A]*[FANA C]*[FANA U]*[FANA |
| | | A]*C*T*A*G*G*C*A*G*A*G*C*[FANA U]*[FANA |
| | | G]*[FANA U]*[FANA U]*[FANA C] |
| SEQ ID NO: 248 | AUM-FANA-FXP3-65 | [FANA A]*[FANA G]*[FANA A]*[FANA U]*[FANA |
| | | G]*[FANA A]*A*G*C*C*T*T*G*G*T*[FANA |
| | | C]*[FANA A]*[FANA G]*[FANA U]*[FANA G]*[FANA |
| | | C] |
| SEQ ID NO: 249 | AUM-FANA-FXP3-66 | [FANA U]*[FANA U]*[FANA U]*[FANA C]*[FANA |
| | | U]*[FANA U]*G*C*G*G*A*A*C*T*C*[FANA |
| | | C]*[FANA A]*[FANA G]*[FANA C]*[FANA U]*[FANA |
| | | C] |
| SEQ ID NO: 250 | AUM-FANA-FXP3-67 | [FANA A]*[FANA C]*[FANA A]*[FANA G]*[FANA |
| | | G]*[FANA U]*G*A*A*T*T*C*T*A*A*[FANA |
| | | C]*[FANA A]*[FANA G]*[FANA G]*[FANA C]*[FANA |
| | | C] |
| SEQ ID NO: 251 | AUM-FANA-FXP3-68 | [FANA U]*[FANA A]*[FANA G]*[FANA U]*[FANA |
| | | U]*[FANA C]*C*T*C*T*G*C*A*G*T*[FANA |
| | | C]*[FANA U]*[FANA A]*[FANA A]*[FANA G]*[FANA |
| | | C] |
| SEQ ID NO: 252 | AUM-FANA-FXP3-69 | [FANA A]*[FANA U]*[FANA A]*[FANA A]*[FANA |
| | | A]*[FANA U]*G*A*G*T*A*G*T*T*C*[FANA |
| | | C]*[FANA U]*[FANA C]*[FANA U]*[FANA G]*[FANA |
| | | C] |
| SEQ ID NO: 253 | AUM-FANA-FXP3-70 | [FANA U]*[FANA U]*[FANA U]*[FANA G]*[FANA |
| | | A]*[FANA G]*T*G*T*A*C*T*G*A*G*[FANA |
| | | G]*[FANA C]*[FANA A]*[FANA G]*[FANA G]*[FANA |
| | | C] |
| SEQ ID NO: 254 | AUM-FANA-FXP3-71 | [FANA G]*[FANA U]*[FANA U]*[FANA C]*[FANA |
| | | A]*[FANA A]*G*G*A*A*G*A*A*G*A*[FANA |
| | | G]*[FANA G]*[FANA A]*[FANA G]*[FANA |
| | | G]*[FANA C] |
| SEQ ID NO: 255 | AUM-FANA-FXP3-72 | [FANA A]*[FANA C]*[FANA U]*[FANA G]*[FANA |
| | | A]*[FANA C]*C*A*A*G*G*C*T*T*C*[FANA |
| | | A]*[FANA U]*[FANA C]*[FANA U]*[FANA G]*[FANA |
| | | U] |
| SEQ ID NO: 256 | AUM-FANA-FXP3-73 | [FANA A]*[FANA A]*[FANA A]*[FANA C]*[FANA |
| | | A]*[FANA G]*C*A*C*A*T*T*C*C*C*[FANA |
| | | A]*[FANA G]*[FANA A]*[FANA G]*[FANA |
| | | U]*[FANA U] |
| SEQ ID NO: 257 | AUM-FANA-FXP3-74 | [FANA U]*[FANA U]*[FANA G]*[FANA U]*[FANA |
| | | G]*[FANA A]*A*G*G*C*T*C*T*G*T*[FANA |
| | | U]*[FANA U]*[FANA G]*[FANA G]*[FANA C]*[FANA |
| | | U] |
| SEQ ID NO: 258 | AUM-FANA-FXP3-75 | [FANA G]*[FANA U]*[FANA U]*[FANA G]*[FANA |
| | | U]*[FANA G]*A*A*G*G*C*T*C*T*G*[FANA |
| | | U]*[FANA U]*[FANA U]*[FANA G]*[FANA |
| | | G]*[FANA C] |
| SEQ ID NO: 259 | AUM-FANA-FXP3-76 | [FANA G]*[FANA U]*[FANA U]*[FANA G]*[FANA |
| | | U]*[FANA U]*T*G*A*G*T*G*T*A*C*[FANA |
| | | U]*[FANA G]*[FANA A]*[FANA G]*[FANA |
| | | G]*[FANA C] |
| SEQ ID NO: 260 | AUM-FANA-FXP3-77 | [FANA A]*[FANA G]*[FANA G]*[FANA U]*[FANA |
| | | G]*[FANA A]*A*T*T*C*T*A*A*C*A*[FANA |
| | | G]*[FANA G]*[FANA C]*[FANA C]*[FANA G]*[FANA |
| | | U] |
| SEQ ID NO: 261 | AUM-FANA-FXP3-78 | [FANA G]*[FANA U]*[FANA C]*[FANA U]*[FANA |
| | | A]*[FANA A]*G*C*T*G*A*G*G*C*A*[FANA |
| | | U]*[FANA G]*[FANA G]*[FANA A]*[FANA |
| | | U]*[FANA C] |
| SEQ ID NO: 262 | AUM-FANA-FXP3-79 | [FANA A]*[FANA G]*[FANA U]*[FANA C]*[FANA |
| | | U]*[FANA A]*A*G*C*T*G*A*G*G*C*[FANA |
| | | A]*[FANA U]*[FANA G]*[FANA G]*[FANA |
| | | A]*[FANA U] |
| SEQ ID NO: 263 | AUM-FANA-FXP3-80 | [FANA G]*[FANA A]*[FANA U]*[FANA G]*[FANA |
| | | A]*[FANA U]*G*C*C*A*C*A*G*A*T*[FANA |
| | | G]*[FANA A]*[FANA A]*[FANA G]*[FANA C]*[FANA |
| | | C] |
| SEQ ID NO: 264 | AUM-FANA-FXP3-81 | [FANA U]*[FANA G]*[FANA U]*[FANA U]*[FANA |
| | | G]*[FANA A]*G*T*G*A*G*G*G*A*C*[FANA |
| | | A]*[FANA G]*[FANA G]*[FANA A]*[FANA |
| | | U]*[FANA U] |
| SEQ ID NO: 265 | AUM-FANA-FXP3-82 | [FANA A]*[FANA A]*[FANA C]*[FANA C]*[FANA |
| | | U]*[FANA C]*A*A*A*G*C*T*G*C*A*T*[FANA |
| | | C]*[FANA A]*[FANA U]*[FANA C]*[FANA |
| | | A]*[FANA ] |
| SEQ ID NO: 266 | AUM-FANA-FXP3-83 | [FANA C]*[FANA A]*[FANA C]*[FANA A]*[FANA |
| | | G]*[FANA G]*T*G*A*A*T*T*C*T*A*[FANA |
| | | A]*[FANA C]*[FANA A]*[FANA G]*[FANA G]*[FANA |
| | | C] |
| SEQ ID NO: 267 | AUM-FANA-FXP3-84 | [FANA A]*[FANA G]*[FANA U]*[FANA C]*[FANA |
| | | U]*[FANA A]*A*G*C*T*G*A*G*G*C*[FANA |
| | | A]*[FANA U]*[FANA G]*[FANA G]*[FANA |
| | | A]*[FANA U] |
| SEQ ID NO: 268 | AUM-FANA-FXP3-85 | [FANA A]*[FANA U]*[FANA G]*[FANA U]*[FANA |
| | | A]*[FANA C]*C*A*A*G*G*C*A*G*G*[FANA |
| | | C]*[FANA U]*[FANA C]*[FANA U]*[FANA U]*[FANA |
| | | C] |
| SEQ ID NO: 269 | AUM-FANA-FXP3-86 | [FANA A]*[FANA A]*[FANA U]*[FANA A]*[FANA |
| | | U]*[FANA U]*A*G*G*A*T*G*G*T*G*[FANA |
| | | U]*[FANA C]*[FANA U]*[FANA G]*[FANA U]*[FANA |
| | | C] |
| SEQ ID NO: 270 | AUM-FANA-FXP3-87 | [FANA U]*[FANA A]*[FANA U]*[FANA A]*[FANA |
| | | G]*[FANA C]*T*G*G*T*T*G*T*G*A*[FANA |
| | | G]*[FANA G]*[FANA G]*[FANA C]*[FANA U]*[FANA |
| | | C] |
| SEQ ID NO: 271 | AUM-FANA-FXP3-88 | [FANA U]*[FANA C]*[FANA A]*[FANA U]*[FANA |
| | | U]*[FANA G]*A*G*T*G*T*C*C*T*C*[FANA |
| | | U]*[FANA G]*[FANA C]*[FANA C]*[FANA U]*[FANA |
| | | C] |
| SEQ ID NO: 272 | AUM-FANA-FXP3-89 | [FANA A]*[FANA C]*[FANA U]*[FANA A]*[FANA |
| | | A]*[FANA C]*A*G*C*T*A*G*A*G*G*[FANA |
| | | C]*[FANA U]*[FANA U]*[FANA U]*[FANA G]*[FANA |
| | | C] |
| SEQ ID NO: 273 | AUM-FANA-FXP3-90 | [FANA C]*[FANA C]*[FANA A]*[FANA C]*[FANA |
| | | U]*[FANA U]*G*C*A*G*A*C*T*C*C*[FANA |
| | | A]*[FANA U]*[FANA U]*[FANA U]*[FANA |
| | | G]*[FANA C] |
| SEQ ID NO: 274 | AUM-FANA-FXP3-91 | [FANA A]*[FANA U]*[FANA A]*[FANA G]*[FANA |
| | | G]*[FANA U]*A*C*A*C*G*T*A*G*G*[FANA |
| | | A]*[FANA C]*[FANA U]*[FANA U]*[FANA G]*[FANA |
| | | C] |
| SEQ ID NO: 275 | AUM-FANA-FXP3-92 | [FANA A]*[FANA U]*[FANA U]*[FANA U]*[FANA |
| | | C]*[FANA A]*T*T*G*A*G*T*G*T*C*[FANA |
| | | C]*[FANA U]*[FANA C]*[FANA U]*[FANA G]*[FANA |
| | | C] |
| SEQ ID NO: 276 | AUM-FANA-FXP3-93 | [FANA U]*[FANA A]*[FANA G]*[FANA A]*[FANA |
| | | U]*[FANA U]*G*A*G*A*C*A*G*A*G*[FANA |
| | | A]*[FANA U]*[FANA G]*[FANA G]*[FANA |
| | | G]*[FANA C] |
| SEQ ID NO: 277 | AUM-FANA-FXP3-94 | [FANA U]*[FANA G]*[FANA A]*[FANA C]*[FANA |
| | | U]*[FANA G]*G*A*T*G*T*A*A*G*T*[FANA |
| | | A]*[FANA U]*[FANA A]*[FANA G]*[FANA |
| | | G]*[FANA C] |
| SEQ ID NO: 278 | AUM-FANA-FXP3-95 | [FANA U]*[FANA G]*[FANA U]*[FANA U]*[FANA |
| | | C] *[FANA A] *A*G*G*T*A*T*G*G*A*[FANA |
| | | G]*[FANA C]*[FANA A]*[FANA G]*[FANA G]*[FANA |
| | | C] |
| SEQ ID NO: 279 | AUM-FANA-FXP3-96 | [FANA A]*[FANA G]*[FANA U]*[FANA U]*[FANA |
| | | C]*[FANA A]*C*G*A*A*T*G*T*A*C*[FANA |
| | | C]*[FANA A]*[FANA A]*[FANA G]*[FANA G]*[FANA |
| | | C] |
| SEQ ID NO: 280 | AUM-FANA-FXP3-97 | [FANA U]*[FANA G]*[FANA U]*[FANA U]*[FANA |
| | | A] *[FANA C]*G*G*G*A*A*T*A*G*G*[FANA |
| | | A]*[FANA G]*[FANA G]*[FANA A]*[FANA |
| | | G]*[FANA C] |
| SEQ ID NO: 281 | AUM-FANA-FXP3-98 | [FANA C]*[FANA U]*[FANA A]*[FANA A]*[FANA |
| | | C]*[FANA A]*G*C*T*A*G*A*G*G*C*[FANA |
| | | U]*[FANA U]*[FANA U]*[FANA G]*[FANA C]*[FANA |
| | | C] |
| SEQ ID NO: 282 | AUM-FANA-FXP3-99 | [FANA U]*[FANA A]*[FANA G]*[FANA G]*[FANA |
| | | U]*[FANA A]*C*A*C*G*T*A*G*G*A*[FANA |
| | | C]*[FANA U]*[FANA U]*[FANA G]*[FANA C]*[FANA |
| | | C] |
| SEQ ID NO: 283 | AUM-FANA-FXP3-100 | [FANA U]*[FANA U]*[FANA U]*[FANA C]*[FANA |
| | | A]*[FANA U]*T*G*A*G*T*G*T*C*C*[FANA |
| | | U]*[FANA C]*[FANA U]*[FANA G]*[FANA C]*[FANA |
| | | C] |
| SEQ ID NO: 284 | AUM-FANA-FXP3-101 | [FANA A]*[FANA U]*[FANA A]*[FANA G]*[FANA |
| | | U]*[FANA U]*A*C*C*C*T*G*G*T*T*[FANA |
| | | U]*[FANA C]*[FANA U]*[FANA A]*[FANA C]*[FANA |
| | | C] |
| SEQ ID NO: 285 | AUM-FANA-FXP3-102 | [FANA U]*[FANA U]*[FANA C]*[FANA U]*[FANA |
| | | A]*[FANA C]*C*A*G*A*T*C*T*T*G*[FANA |
| | | U]*[FANA C]*[FANA G]*[FANA G]*[FANA A]*[FANA |
| | | C] |
| SEQ ID NO: 286 | AUM-FANA-FXP3-103 | [FANA U]*[FANA U]*[FANA C]*[FANA U]*[FANA |
| | | A]*[FANA G]*C*A*C*T*C*T*C*T*T*[FANA |
| | | U]*[FANA C]*[FANA A]*[FANA U]*[FANA U]*[FANA |
| | | U] |
| SEQ ID NO: 287 | AUM-FANA-FXP3-104 | [FANA A]*[FANA A]*[FANA G]*[FANA U]*[FANA |
| | | A]*[FANA U]*A*G*G*C*A*C*G*T*G*[FANA |
| | | A]*[FANA G]*[FANA U]*[FANA G]*[FANA |
| | | U]*[FANA U] |
| SEQ ID NO: 288 | AUM-FANA-FXP3-105 | [FANA U]*[FANA C]*[FANA A]*[FANA C]*[FANA |
| | | U]*[FANA A]*C*T*A*G*G*C*A*G*A*[FANA |
| | | G]*[FANA C]*[FANA U]*[FANA G]*[FANA U]*[FANA |
| | | U] |
| SEQ ID NO: 289 | AUM-FANA-FXP3-106 | [FANA A]*[FANA C]*[FANA A]*[FANA A]*[FANA |
| | | C]*[FANA U]*A*A*C*A*G*C*T*A*G*[FANA |
| | | A]*[FANA G]*[FANA G]*[FANA C]*[FANA U]*[FANA |
| | | U] |
| SEQ ID NO: 290 | AUM-FANA-FXP3-107 | [FANA A]*[FANA C]*[FANA A]*[FANA C]*[FANA |
| | | U]*[FANA C]*A*C*G*T*G*C*C*T*A*[FANA |
| | | U]*[FANA A]*[FANA C]*[FANA U]*[FANA U]*[FANA |
| | | A] |
| SEQ ID NO: 291 | AUM-FANA-FXP3-108 | [FANA U]*[FANA U]*[FANA C]*[FANA A]*[FANA |
| | | U]*[FANA U]*T*G*G*T*A*T*C*C*G*[FANA |
| | | C]*[FANA U]*[FANA U]*[FANA U]*[FANA C]*[FANA |
| | | U] |
| SEQ ID NO: 292 | AUM-FANA-FXP3-109 | [FANA A]*[FANA G]*[FANA U]*[FANA G]*[FANA |
| | | A]*[FANA G]*A*C*G*T*G*G*G*G*T*[FANA |
| | | A]*[FANA C]*[FANA A]*[FANA U]*[FANA C]*[FANA |
| | | U] |
| SEQ ID NO: 293 | AUM-FANA-FXP3-110 | [FANA U]*[FANA G]*[FANA G]*[FANA U]*[FANA |
| | | A]*[FANA U]*C*C*G*C*T*T*T*C*T*[FANA |
| | | C]*[FANA C]*[FANA U]*[FANA G]*[FANA C]*[FANA |
| | | U] |
| SEQ ID NO: 294 | AUM-FANA-FXP3-111 | [FANA U]*[FANA U]*[FANA C]*[FANA A]*[FANA |
| | | U]*[FANA U]*G*A*G*T*G*T*C*C*T*[FANA |
| | | C]*[FANA U]*[FANA G]*[FANA C]*[FANA C]*[FANA |
| | | U] |
| SEQ ID NO: 295 | AUM-FANA-FXP3-112 | [FANA A]*[FANA C]*[FANA U]*[FANA A]*[FANA |
| | | G]*[FANA U]*A*A*A*G*G*G*T*G*G*[FANA |
| | | U]*[FANA C]*[FANA U]*[FANA U]*[FANA A]*[FANA |
| | | U] |
| SEQ ID NO: 296 | AUM-FANA-FXP3-113 | [FANA A]*[FANA U]*[FANA A]*[FANA G]*[FANA |
| | | U]*[FANA G]*C*C*C*G*T*G*G*T*T*[FANA |
| | | C]*[FANA C]*[FANA A]*[FANA G]*[FANA A]*[FANA |
| | | U] |
| SEQ ID NO: 297 | AUM-FANA-FXP3-114 | [FANA U]*[FANA G]*[FANA U]*[FANA U]*[FANA |
| | | C]*[FANA Ul*A*G*C*A*C*T*C*T*C*[FANA |
| | | U]*[FANA U]*[FANA U]*[FANA C]*[FANA A]*[FANA |
| | | U] |
| SEQ ID NO: 298 | AUM-FANA-FXP3-115 | [FANA U]*[FANA A]*[FANA U]*[FANA U]*[FANA |
| | | A]*[FANA G]*G*A*T*G*G*T*G*T*C*[FANA |
| | | U]*[FANA G]*[FANA U]*[FANA C]*[FANA A]*[FANA |
| | | U] |
| SEQ ID NO: 299 | AUM-FANA-FXP3-116 | [FANA C]*[FANA A]*[FANA A]*[FANA U]*[FANA |
| | | A]*[FANA C]*C*T*C*T*C*T*G*C*CIFANA |
| | | A]*[FANA C]*[FANA U]*[FANA U]*[FANA U]*[FANA |
| | | C] |
| SEQ ID NO: 300 | AUM-FANA-FXP3-117 | [FANA C]*[FANA U]*[FANA A]*[FANA A]*[FANA |
| | | G]*[FANA A]*A*G*G*A*T*G*A*T*G*[FANA |
| | | C]*[FANA U]*[FANA G]*[FANA U]*[FANA U]*[FANA |
| | | C] |
| SEQ ID NO: 301 | AUM-FANA-FXP3-118 | [FANA C]*[FANA A]*[FANA C]*[FANA U]*[FANA |
| | | A]*[FANA C]*T*A*G*G*C*A*G*A*G*[FANA |
| | | C]*[FANA U]*[FANA G]*[FANA U]*[FANA U]*[FANA |
| | | C] |
| SEQ ID NO: 302 | AUM-FANA-FXP3-119 | [FANA G]*[FANA G]*[FANA U]*[FANA U]*[FANA |
| | | G]*C*T*G*T*C*T*T*T*C*C*T*[FANA G]*[FANA |
| | | G]*[FANA G]*[FANA U]*[FANA G] |
| SEQ ID NO: 303 | AUM-FANA-5B | [FANA G][FANA G][FANA U*][FANA U*][FANA |
| | | G]C*T*G*T*C*T*T*T*C*C*T*[FANA G][FANA |
| | | G][FANA G][FANA U*][FANA G] |
| SEQ ID NO: 304 | AUM-FANA-6B | [FANA A*][FANA G*][FANA U*][FANA U*][FANA |
| | | G]C*T*G*C*T*T*T*A*G*G*T*[FANA G*][FANA |
| | | G*][FANA A*][FANA G*][FANA U] |

[FANA A, U, C, G]: FANA Modified Base; A, T, G, C: unmodified DNA base; * - Phosphorothioate Linkage

Non-limiting examples of modified AONs according to the embodiments described herein can include, but are not limited to, any of the sequences SEQ ID NOs 1-9, SEQ ID NOs:11-19, SEQ ID NOs: 21-29, SEQ ID NOs: 31-138, SEQ ID NOs: 139-192, or SEQ ID NOs: 193-302, in any of the disposition recited in the formulas in Table 1.

In another embodiment, the invention provides a pharmaceutical composition including a modified antisense oligonucleotide (AON) including at least one 2′-deoxy-2′-fluoro-β-D-arabinonucleotide (2′-FANA modified nucleotide) and a pharmaceutically acceptable carrier, wherein the AON binds to a Foxp3 mRNA.

By “pharmaceutically acceptable” it is meant the carrier, diluent or excipient must be compatible with the other ingredients of the formulation and not deleterious to the recipient thereof. For example, the carrier, diluent, or excipient or composition thereof may not cause any undesirable biological effects or interact in an undesirable manner with any of the other components of the pharmaceutical composition in which it is contained.

In various aspects, the AON is a hybrid chimera AON including at least one 2′-FANA modified nucleotide and at least one unmodified deoxyribonucleotide, wherein the AON is a 2′-FANA AON. In some aspects, the 2′-FANA AON includes from about 0 to about 20 2′-deoxy-2′-fluoro-β-D-arabinonucleotide at the 5′-end and from about 0 to about 20 2′-deoxy-2′-fluoro-β-D-arabinonucleotide at the 3′-end, flanking a sequence including from about 0 to about 20 deoxyribonucleotide residues. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs 1-9, SEQ ID NOs:11-19, SEQ ID NOs: 21-29, SEQ ID NOs: 31-138, SEQ ID NOs: 139-192, or SEQ ID NOs: 193-302, or a sequence complimentary thereto.

In an additional embodiment, the invention provides a method of reducing the expression level of Foxp3 gene in a cell including contacting the cell with at least one antisense oligonucleotide (AON), wherein the AON binds to Foxp3 mRNA, and wherein the AON includes at least one 2′-deoxy-2′-fluoro-β-D-arabinonucleotide (2′-FANA modified nucleotide).

As used herein “at least one” refers to the administration of one or more 2′-FANA AONs to increase and/or enhance the desired effect. To reach such effect, 1, 2, 3, 4, 5, 6, 7, 8, 9 or 10 different 2′-FANA AONs can be administered to the same subject, aiming at synergizing the effects. The at least one 2′-FANA AON, also referred to as “a plurality or 2′-FANA AONs” can be administered one at a time, several at a time, or all at a time, depending the sought-after outcome, the subject's physical state, or any other parameter that could affect the administration schedule, such as the evolution or progression or a disease state.

In one aspect 2, 3, 4, 5, 6, 7, 8, 9 or 10 AONs are contacted with the cell.

The synthetic AON can be delivered in any suitable way to permit contact and uptake of the AON by the cell, and can do so without the need for a delivery vehicle or transfection agent. In some embodiments, the delivery method includes a transfection technique, including but not limited to, electroporation or microinjection. In other embodiments, the delivery method is gymnotic. Cells can be contacted with AONs along with a transfection agent or delivery vehicle or other transfection method. Non-limiting examples of transfection reagents and methods include: gene gun, electroporation, nanoparticle delivery (e.g. PEG-coated nanoparticles), cationic lipids and/or polymers, zwitterionic lipids and/or polymers, neutral lipids and/or polymers. Specific examples include: in vivo-jetPEI, X-tremeGENE reagents, DOPC neutral liposome, cyclodextrin-containing polymer CAL101, and lipid nanoparticles.

In one embodiment, the cell is one in a population of in vitro cultured cells. In another embodiment, the cell is part of a population in a living host or subject. For example, an AON may be delivered to a cell in an in vivo environment for the purposes of silencing FOXP3 gene expression the cell. Additionally, an AON may be delivered to a cultured cell in order to study its effect on the cell type in question.

As used herein “reducing the expression level of Foxp3 gene” refers to any change in the expression level of the Foxp3 gene, that is lower that the expression level before the cell was contacted with the AON. The phrase “expression level of Foxp3” is meant to refer to both protein and mRNA expression levels without distinction.

In one aspect, the cell is a regulatory T cell (Treg).

The term “Treg” is used interchangeably with “regulatory T cell” or “suppressor T cells.” It has general meaning in the art and refers to a subset of T helper cells that modulate the immune system, maintain tolerance to self-antigens, and prevent autoimmune disease. Tregs are immunosuppressive cells and generally suppress or downregulate induction and proliferation of effector T cells. Tregs express the biomarkers CD4, FOXP3, and CD25 and are thought to be derived from the same lineage as naive CD4 cells.

The immune system must be able to discriminate between self and non-self. When self/non-self-discrimination fails, the immune system can either destroys cells and tissues of the body which causes autoimmune diseases, or fails to destroy abnormal cells such as cancer cells which leads to anti-tumor immunity suppression.

Regulatory T cells actively suppress activation of the immune system and prevent pathological self-reactivity, i.e. autoimmune disease. The critical role regulatory T cells play within the immune system is evidenced by the severe autoimmune syndrome that results from a genetic deficiency in regulatory T cells, as well as by the observed excess of regulatory T cell activity that prevents the immune system from destroying cancer cells. Indeed, Tregs tend to be unregulated in individuals with cancer, and they seem to be recruited to the site of many tumors. Studies in both humans and animal models have implicated that high numbers of Tregs in the tumor microenvironment is indicative of poor prognosis, and Tregs are thought to suppress tumor immunity, thus hindering the body's innate ability to control the growth of cancer cells.

Regulatory T cells can produce Granzyme B, which in turn can induce apoptosis of effector T cells. Another major mechanism of suppression by regulatory T cells is through the prevention of co-stimulation through the CD28 receptor, expressed at on effector T cells' surface, by the action of the molecule CTLA-4.

In various aspects, the Treg expresses the cellular markers CD4 and CD25.

The phrase “molecular marker” is used alternatively with the phrases “cellular marker”, “cell surface marker” or “cell surface protein” and refers to any protein that is expressed at the surface of a cell, and that can be, for example, used to differentiate one cell type from another.

“Polypeptide” or “protein” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides, typically over 100 amino acids. The term “peptide” typically refers to short polypeptides, typically under 100 amino acids.

In other aspects, the AON is a hybrid chimera AON including at least one 2′-FANA modified nucleotide and at least one unmodified deoxyribonucleotide, wherein the AON is a 2′-FANA AON. In various aspects, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs 1-9, SEQ ID NOs:11-19, SEQ ID NOs: 21-29, SEQ ID NOs: 31-138, SEQ ID NOs: 139-192, or SEQ ID NOs: 193-302, or a sequence complimentary thereto.

In another embodiment, the invention provides a method of increasing anti-tumor immunity in a subject in need thereof including administering to the subject at least one antisense oligonucleotide (AON), wherein the AON binds to a Foxp3 mRNA, and wherein the AON includes at least one 2′-deoxy-2′-fluoro-β-D-arabinonucleotide (2′-FANA modified nucleotide). In one aspect 2, 3, 4, 5, 6, 7, 8, 9 or 10 AONs are administered to the subject.

As used herein, “anti-tumor immunity” refers to the immune response that has an anti-tumor effect, i.e. targeting tumor/cancer cells to help the body fight against cancer. Anti-tumor immunity relies on both innate and acquired immunity.

The term “immune response” refers to an integrated bodily response to an antigen and preferably refers to a cellular immune response or a cellular as well as a humoral immune response. The immune response may be protective/preventive/prophylactic and/or therapeutic.

The cellular response relates to cells called T cells or T-lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, and prevent the production of more diseased cells. The humoral response relates to B cells or B lymphocytes, which are a type of lymphocyte of the adaptive immune system and are important for immune surveillance. The B cell antigen-specific receptor is an antibody molecule on the B cell surface that recognizes whole pathogens without any need for antigen processing. Each lineage of B cell expresses a different antibody, so the complete set of B cell antigen receptors represent all the antibodies that an individual can generate.

The AON of the invention is designed to target a portion of the Foxp3 mRNA expression in Treg cells to decrease Treg function and increase antitumor immunity. This increase in antitumor immunity may aid in treating the patient. Thus, in some embodiments, at least a portion of the 2′-FANA AON is complementary to part of an mRNA sequence that corresponds to the Foxp3 gene. The 2′-FANA AON may be designed to target and bind to all or a portion of the Foxp3 mRNA.

The term “subject” as used herein refers to any individual or patient to which the subject methods are performed. Generally the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The terms “administration of” and or “administering” should be understood to mean providing a pharmaceutical composition in a therapeutically effective amount to the subject in need of thereof to achieve a desired outcome.

In one aspect, the AON decreases the activity of a regulatory T cell (Treg). In some aspects, the Treg expresses the cellular markers CD4 and CD25. In various aspects, the AON induces Treg apoptosis.

In another aspect, the AON increases the activity of an immune cell. In certain aspects, the immune cell is CD8⁺ T cell, CD4⁺ T cell, B cell, natural killer cell, macrophage, dendritic cell or a combination thereof.

The terms “immunoreactive cell”, “immune cells”, or “immune effector cells” in the context of the present invention relate to a cell which exerts effector functions during an immune reaction. An “immune cell” preferably is capable of binding an antigen or a cell characterized by presentation of an antigen or an antigen peptide derived from an antigen and mediating an immune response. For example, such cells secrete cytokines and/or chemokines, secrete antibodies, recognize cancerous cells, and optionally eliminate such cells. For example, immune cells comprise T cells (cytotoxic T cells, helper T cells, tumor infiltrating T cells), B cells, natural killer cells, neutrophils, macrophages, and dendritic cells.

In various aspects, the AON is a hybrid chimera AON including at least one 2′-FANA modified nucleotide and at least one unmodified deoxyribonucleotide, wherein the AON is a 2′-FANA AON. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs 1-9, SEQ ID NOs:11-19, SEQ ID NOs: 21-29, SEQ ID NOs: 31-138, SEQ ID NOs: 139-192, or SEQ ID NOs: 193-302 or a sequence complimentary thereto.

In yet another embodiment, the invention provides a method of treating cancer in a subject in need thereof including administering to the subject at least one antisense oligonucleotide (AON), wherein the AON binds to a Foxp3 mRNA, and wherein the AON includes at least one 2′-deoxy-2′-fluoro-β-D-arabinonucleotide (2′-FANA modified nucleotide). In one aspect 2, 3, 4, 5, 6, 7, 8, 9 or 10 AONs are administered to the subject.

The term “treatment” is used interchangeably herein with the term “therapeutic method” and refers to both 1) therapeutic treatments or measures that cure, slow down, lessen symptoms of, and/or halt progression of a diagnosed conditions or disorder, and 2) and prophylactic/preventative measures. Those in need of treatment may include individuals already having a particular medical disorder as well as those who may ultimately acquire the disorder (i.e., those needing preventive measures). To “treat” a disease as the term is used herein, means to reduce the frequency of the disease or disorder, reducing the frequency with which a symptom of the one or more symptoms disease or disorder is experienced by the subject.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome. Such amount should be sufficient to a beneficial effect to the subject to which the compound is administered. The effective amount can be determined as described herein. As used herein, a “therapeutically effective amount” is the amount of cells which are sufficient to provide a beneficial effect to the subject to which the cells are administered.

The terms “administration of” and or “administering” should be understood to mean providing a pharmaceutical composition in a therapeutically effective amount to the subject in need of treatment. Administration route is not specifically limited and can include oral, intravenous, intramuscular, infusion, intrathecal, intradermal, subcutaneous, sublingual, buccal, rectal, vaginal, ocular, otic route, nasal, inhalation, nebulization, cutaneous, topical, transdermal, intraperitoneal or intratumoral administrations.

The term “cancer” refers to a group diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure to environmental pollutants, tobacco and or alcohol use, obesity, poor diet, lack of physical activity or any combination thereof. “Cancer cell” or “tumor cell”, and grammatical equivalents refer to the total population of cells derived from a tumor or a pre-cancerous lesion, including both non tumorigenic cells, which comprise the bulk of the tumor population, and tumorigenic stem cells (cancer stem cells).

Exemplary cancers include, but are not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland'Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In certain aspects, the cancer breast, liver, ovarian, pancreatic, lung cancer, melanoma or glioblastoma.

In one aspect, the cancer is lung cancer.

As used herein, “lung cancer” is meant to include, but not to be limited to, all type of lung cancer at all stages of progression, which encompasses lung carcinoma; metastatic lung cancer; the three main forms of non-small cell lung carcinoma (NSCLC), i.e. lung adenocarcinoma, squamous cell carcinoma and large cell carcinoma; small cell lung cancer (SCLC) and mesothelioma.

In various aspects, the AON reduces expression level of a Foxp3 gene. In some aspects, the AON is a hybrid chimera AON including at least one 2′-FANA modified nucleotide and at least one unmodified deoxyribonucleotide, and wherein the AON is a 2′-FANA AON. In one aspect, the 2′-FANA AON includes at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs 1-9, SEQ ID NOs:11-19, SEQ ID NOs: 21-29, SEQ ID NOs: 31-138, SEQ ID NOs: 139-192, or SEQ ID NOs: 193-302, or a sequence complimentary thereto.

In various aspects, the 2′-FANA AON increases anti-tumor immunity in the subject. In some aspects, the 2′-FANA AON decreases the activity of a regulatory T cell (Treg) and/or increases the activity of an immune cell.

In certain aspects, the AON further includes a pharmaceutically acceptable carrier. In other aspects, an immunotherapeutic agent and/or a chemotherapeutic agent is further administered.

The term “immune modulator” or “immunotherapeutic agent” as used herein refers to any therapeutic agent that modulates the immune system. Examples of immune modulators include eicosanoids, cytokines, prostaglandins, interleukins, chemokines, checkpoint regulators, TNF superfamily members, TNF receptor superfamily members and interferons. Specific examples of immune modulators include PGI2, PGE2, PGF2, CCL14, CCL19, CCL20, CCL21, CCL25, CCL27, CXCL12, CXCL13, CXCL-8, CCL2, CCL3, CCL4, CCL5, CCL11, CCL26, CXCL7, CXCL10, ILL IL2, IL3, IL4, IL5, IL6, IL7, IL8, IL9, IL10, IL11, IL12, IL13, IL15, IL17, IL17, INF-α, INF-β, INF-ϵ, INF-γ, G-CSF, TNF-α, CTLA, CD20, PD1, PD1L1, PD1L2, ICOS, imiquimod, CD200, CD52, LTα, LTαβ, LIGHT, CD27L, 41BBL, FasL, Ox40L, April, TL1A, CD3OL, TRAIL, RANKL, BAFF, TWEAK, CD40L, EDA1, EDA2, APP, NGF, TNFR1, TNFR2, LTβR, HVEM, CD27, 4-1BB, Fas, Ox40, AITR, DR3, CD30, TRAIL-R1, TRAIL-R2, TRAIL-R3, TRAIL-R4, RANK, BAFFR, TACI, BCMA, Fn14, CD40, EDAR XEDAR, DR6, DcR3, NGFR-p75, and Taj. Other examples of immune modulators include specific antibodies such as Actemra (tocilizumab), Cimzia (CDP870), Enbrel (enteracept), Kineret, abatacept (Orencia), Remicade (infliximab), Rituxan (rituzimab), Simponi (golimumab), Avonex, Rebif, ReciGen, Plegridy, Betaseron, Copaxone, Novatrone, Tysabri (natalizumab), Gilenya (fingolimod), Aubagio (teriflunomide), BG12, Tecfidera, Campath, Lemtrada (alemtuzumab), panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Raptiva (efalizumab), Tysabri (natalizumab), Zenapax (dacliximab), NeutroSpec (Technetium (99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab, Gilotrif (afatinib), Lynparza (olaparib), Perjeta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), Avelumab (Bavencio), Durvalumab (Imfinzi), Iplimumab (Yervoy) and Tarceva (erlotinib).

The term “chemotherapeutic agent”, as used herein, refers to any therapeutic agent having antineoplastic effect used to treat cancer. Chemotherapeutic and antineoplastic agents are well known cytotoxic agents, and include: (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

In certain aspects, the immunotherapeutic agent and/or chemotherapeutic agent is a checkpoint inhibitor, vaccine, chimeric antigen receptor (CAR)-T cell therapy, anti-PD-1 antibody (Nivolumab or Pembrolizumab), anti-PD-L1 antibody (Atezolizumab, Avelumab or Durvalumab) or a combination thereof.

“Checkpoint inhibitor” refers to a therapy for cancer treatment that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), MR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells). Nivolumab and Pembrolizumab are two commercialized anti-PD-1 antibodies approved by the FDA for cancer treatment.

PD-1 has two ligands, PD-L1 and PD-L2, which are members of the B7 family. PD-L1 protein is unregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines. Atezolizumab, Avelumab and Durvalumab are three commercialized anti-PD-L1 antibodies approved by the FDA for cancer treatment.

The term “vaccine” refers to a biological preparation that provides active acquired immunity to a particular disease. A vaccine typically contains an agent that resembles a disease-causing agent and is often made from weakened or killed forms of it, its toxins, or one of its surface proteins. Vaccine stimulates the immune system to recognize the agent as a threat, destroy it, and to further recognize and destroy it in the future. Vaccines can be prophylactic or therapeutic (e.g., cancer vaccines). The term “cancer vaccine” refers to any preparation capable of being used as an inoculation material or as part of an inoculation material, that will provide a treatment for, inhibit and/or convey immunity to cancer and/or tumor growth. A vaccine may be a peptide vaccine, a DNA vaccine or a RNA vaccine.

Immunotherapies include the use of adoptive transfer of genetically engineered T cells, modified to recognize and eliminate cancer cells specifically. T cells can be genetically modified to stably express on their surface chimeric antigen receptors (CAR). CAR are synthetic proteins comprising a signaling endodomain, consisting of an intracellular domain, a transmembrane domain, and an extracellular domain. Upon interaction with the target cancer cell expressing the antigen, the chimeric antigen receptor triggers an intracellular signaling leading to T-cell activation and to a cytotoxic immune response against tumor cells. Such therapies have been found that also bind, and have been shown to be efficient against relapsed/refractory disease. Additionally, CAR-T cells can be engineered to include co-stimulatory receptor that enhance the T-cell-mediated cytotoxic activity. Furthermore, CAR-T cells can be engineered to produce and deliver protein of interest in the tumor microenvironment.

In other aspects, the immunotherapeutic agent and/or chemotherapeutic agent is administered prior to, simultaneously with, or after the administration of the AON.

In certain aspects, a radiotherapy is further administered. In certain aspects, the radiotherapy is administered prior to, simultaneously with, or after the administration of the AON.

Several cancer treatments can be used in “combination therapy”, or “in combination”. The phrases “combination therapy”, “combined with” and the like refer to the use of more than one medication or treatment simultaneously to increase the response. The AON of the present invention might for example be used in combination with other drugs or treatment in use to treat cancer. Specifically the administration of AON to a subject can be in combination with chemotherapy, radiation, or administration of a therapeutic antibody for example. Such therapies can be administered prior to, simultaneously with, or following administration of the AON of the invention.

Presented below are examples discussing hybrid chimera antisense oligonucleotide including deoxyribonucleotide and 2′-deoxy-2′-fluoro-β-D-arabinonucleotide, which binds to a Foxp3 mRNA, contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Materials and Methods

Antibodies and flow cytometry. Commercially available conjugated monoclonal antibodies (mAbs) were used for flow cytometry (BD Pharmingen). Anti-Foxp3 mAb was FJK-16 s (eBioscience), and β-actin antibodies were rabbit mAbs (Cell Signaling). Flow cytometry was performed on a Cyan flow cytometer (Beckman Coulter), and data were analyzed with FlowJo 8 software (Tree-Star). CD4⁺YFP⁺(Foxp3⁺) and CD4⁺YFP⁻(Foxp3⁻) cells were sorted from age- and sex-matched Foxp3YFP-cre mice using a FACS Aria cell sorter (BD Bioscience, UPenn Cell Sorting Facility).

Spleen and peripheral lymph nodes were harvested and processed to single cell suspensions of lymphocytes. Magnetic beads (Miltenyi Biotec, San Diego, Calif.) were used for isolation of conventional T cells (Tconv, CD4⁻CD25⁻) and Treg (CD4⁺CD25⁺) cells. For cell sorting, lymphocytes were isolated from Foxp3^{cre}YFP mice and purified based on CD4 expression as above. Then, CD4⁺YFP+(Foxp3⁺) and CD4⁺YFP⁻ cells were sorted via a FACS Aria cell sorter (BD Bioscience, UPenn Cell Sorting Facility). Cells of interest were analyzed using surface markers, and for Foxp3 staining, surface marker-stained cells were fixed, permeabilized, and labeled with Foxp3-specific mAb. All flow cytometry data was captured using Cyan (Dako) as well as Cytoflex (Beckman Coulter, Brea, Calif.) and analyzed using the FlowJo 10.1r5 software. Data were pooled and shown in histogram as percent of maximum (% of max), a normalization of overlaid data representing number of cells in each bin divided by the number of cells in the bin that contained the largest number of cells.

PrimeFlow assay to study Foxp3 expression (to differentiate Foxp3⁺ cells from Foxp3⁻ cells). PrimeFlow allowed simultaneous measurement of mRNA and protein by flow cytometry. PrimeFlow RNA Assay (Affymetrix) was used according to the manufacturer's instructions, except for an incubation of cells with FOXP3 mAb which was for 1 hour instead of the recommended 30 minutes.

qPCR for Foxp3 mRNA expression. RNA from Foxp3⁺ Treg or Foxp3⁻ TE cells, freshly isolated from pooled lymph node and spleen samples, or isolated and activated with CD3/28 mAb-coated beads (Invitrogen), was obtained using RNeasy Kits (Qiagen). cDNA was synthesized with TaqMan reverse transcription reagents (Applied Biosystems). qPCR was performed using TaqMan Universal PCR Master Mix (Applied Biosystems), and specific primers from Applied Biosystems, and gene expression data were normalized to 18S RNA.

Treg Suppression Assays. CD4⁺ CD25⁻ T-effector (TE) and CD4⁺ CD25⁺ Treg cells were isolated from Foxp3^{YFP-cre} mice using CD4⁺ CD25⁺ Treg isolation kits (130-091-041, Miltenyi Biotec). Cell Trace Violet-labeled or CFSE-labeled Teff cells (5×10⁵) were stimulated with CD3 mAb (5 μg/ml) in the presence of 5×10⁵ irradiated syngeneic T-cell depleted splenocytes (130-049-101, Miltenyi Biotec) and varying ratios of Tregs. After 72 h, proliferation of TE cells was determined by analysis of Cell Trace Violet dilution or CFSE dilution.

Briefly, CD4⁺ CD25⁺ Tregs, were isolated by magnetic beads (Miltenyi Biotec), incubated with CFSE-labeled HD PBMCs at 1:1 to 1:16 Treg/PBMC ratios for 4 days. Cells were then stimulated with CD3 mAb-coated microbeads at a ratio of 3.6 beads/cell. Suppressive function was counted as area under the curves. 3 LC (tumor) and 2 HD Tregs, were used, gradually diluted with their own CD4⁺FOXP3⁻ Teffs (40%-100% Tregs in the mix) and tested in suppression assays to determine how Tregs lost suppressive function with decreased FOXP3⁺ purity after isolation. These data were used for regression analysis, and the resulting equations were applied to adjust results of suppression assays according to exact FOXP3⁺ purity of each isolated Treg sample.

Mice dosing regimen. Three different kinds of in vivo experiments were performed. In some experiments, mice were treated three times with 10 mg/kg FANA. In other experiments, mice were treated daily for a week with 50 mg/kg FANA. In yet some other experiments, using TC1 tumor models, mice received 50 mg/kg FANA daily for two weeks.

Cell Lines and Tumor Models. TC1 cells were derived from mouse lung epithelial cells that were immortalized with HPV-16 E6 and E7, and transformed with the c-Ha-RAS oncogene. For tumor studies, each mouse was shaved on their right flank and injected subcutaneously with 1.2×10⁶ TC1 tumor cells. Tumor volume was determined by the formula: (3.14×long axis×short axis×short axis)/6.

In vivo TC1 tumor model experiment. C57BL/6 mice (The Jackson Laboratory) were inoculated with TC1 tumor cells and mice were divided into 3 groups (10/group) at 7 days. Group 1: Scramble 50 mg/kg; group 2: AUM-FANA-5 50 mg/kg; group 3: AUM-FANA-6 50 mg/kg. Oligonucleotides were dissolved in PBS (10 mg/ml) and 0.1 ml (1 mg) was given intraperitoneally every day for 14 days. Tumor sizes were measured using calipers and tumor volume calculated twice a week, and at the end of the experiment, tumor, draining lymph nodes and spleen were harvested for further analysis.

### Example 2

### Evaluation of Effect of Foxp3 FANAs on the Number of Foxp3 Expressing Cells

The effect of Foxp3 FANAs on the number of Foxp3 expressing cells was evaluated by flow cytometry.

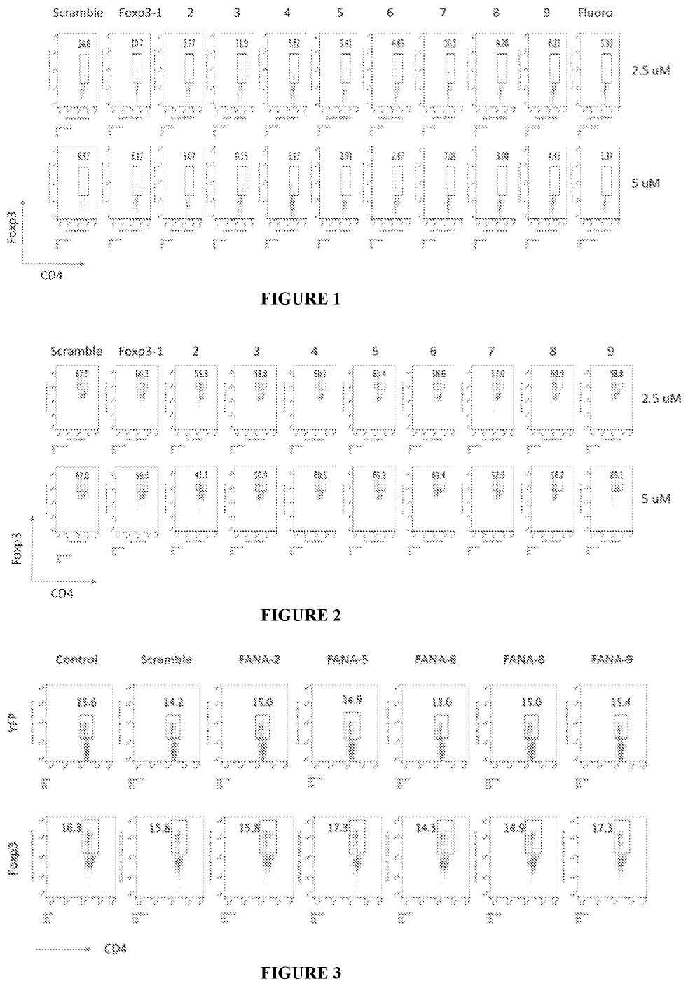

Splenocytes were treated in vitro with CD3 mAb and different FANA sequences for 3 days. As illustrated in FIG. 1, the control scramble FANA indicated that in the cell population isolated from the spleen there was 14.8% of Foxp3 expressing cells, when the FANA concentration was 2.5 μM and 9.57% of Foxp3 expression at 5 μM. Treatment with Foxp3 FANA sequences reduced the number of Foxp3 expressing cells. For example, using AUM-FANA-6, the percentage of cells decreased to 4.63% at 2.5 μM and to 2.97% at 5 μM respectively.

A purified population of Treg cells was independently treated in vitro with CD3/CD28 beads in the presence of 10 U/ml of IL-2 and with several FANA sequences for three days. As illustrated in FIG. 2, the control scramble FANA indicated that 67.5% of the Treg were Foxp3 expressing cells in the 2.5 μM dose case, and 67% in the 5 μM dose. A reduction in the percentage of cells that were positive for Foxp3 expression, as a result of a treatment with a FANA, and as compared to the Scramble control sequence was an indication of a silencing of Foxp3. Many of the sequences lowered the number of Foxp3 expressing cells.

The effect of Foxp3 FANAs on the number of Foxp3 expressing cells was also assessed in vivo. Mice received three 10 mg/kg doses (300 μg into 30 g mice) of FANAs, and 24 hours after the final dose relevant lymphoid tissues were harvested.

As illustrated in FIG. 3, three doses of 10 mg/kg modestly reduced the number of Foxp3 expressing cells in vivo. This dose (10 mg/kg) was much lower than the dose used in tumor models (50 mg/kg), but was included to establish a range. At this dose, AUM-FANA-6 and possibly FANA-8 displayed decreased YFP and/or Foxp3 detection by a small amount (2.6-3%).

### Example 3

### Evaluation of the Cellular Uptake of FANA Oligonucleotides

The in vivo uptake of FANAs was evaluated 24 hours after the injection of 10 mg/kg of fluorescently (APC) labeled scrambled oligonucleotide into mice. Cells were harvested from the spleen, lymph nodes, and blood and were analyzed by flow cytometry.

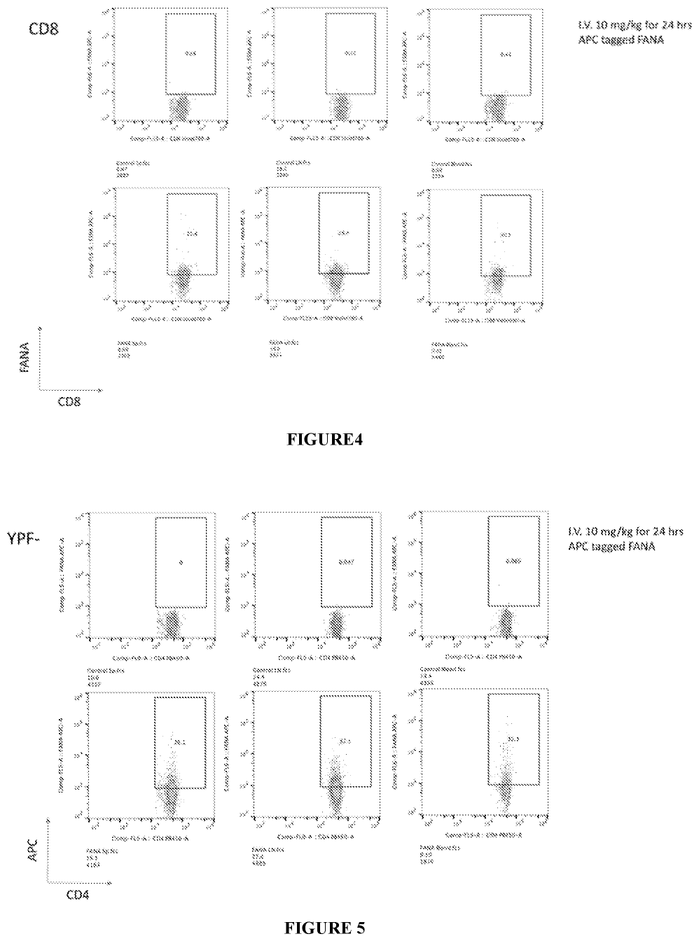

By following CD8 and APC expression by the cells, CD8⁺ and CD8⁻ cells were analyzed. As illustrated in FIG. 4, FANA signal was detected significantly in CD8 cells of all three locations, indicating successful in vivo transfection of CD8 cells, and the in vivo uptake of FANAs by CD8⁺ cells.

Using a mice model that expressed Foxp3 tagged with Yellow Fluorescent Protein (YFP), non-Tregs cells were analyzed, as YFP⁻ (Foxp3⁻) cells, and the uptake of labeled FANAs by non-Tregs cells was assessed. As illustrated in FIG. 5, FANA signal was detected significantly in cells of all three locations (spleen, lymph nodes, and blood) that did not express Foxp3, indicating FANA uptake by non-Treg cells.

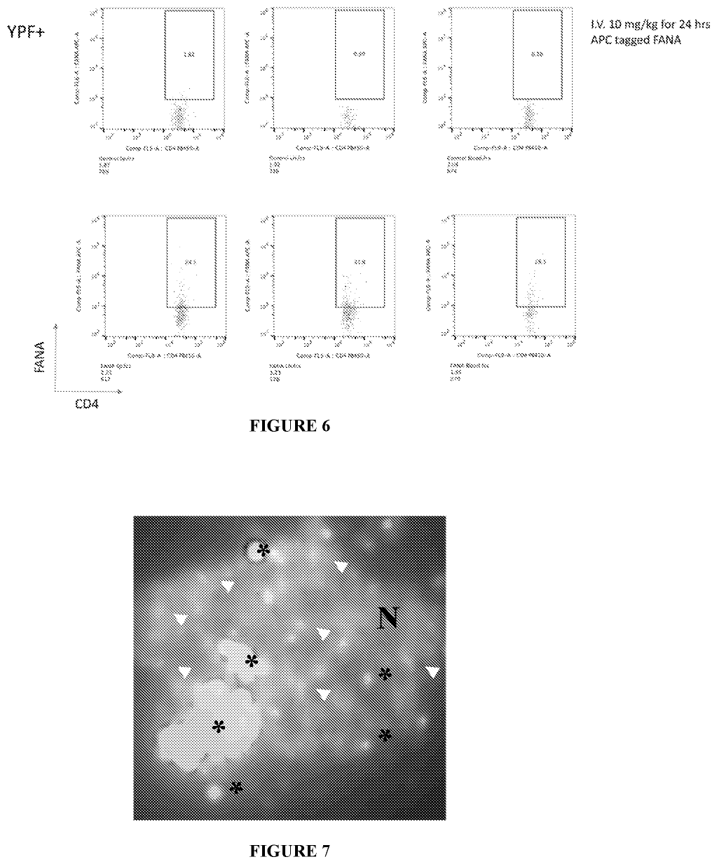

Using this same mice model, expressing Foxp3 tagged YFP, Tregs cells were specifically analyzed, as YFP⁺ (Foxp3⁺) cells, and the uptake of labeled FANAs by Tregs cells was assessed. As illustrated in FIG. 6, FANA signal was detected significantly in Foxp3 expressing (YFP⁺) cells of all three locations (spleen, lymph nodes, and blood), indicating FANA uptake by Treg cells.

In vitro FANA uptake was also evaluated by confocal microscopy. As illustrated in FIG. 7, labeled FANAs were detected in Foxp3 expressing Treg, demonstrating Treg cell uptake. Foxp3 as labeled by the white arrowheads, and FANA as labeled by the black stars, were detected in the nucleus (N) of the cells. The co-localization of Foxp3 and FANAs in the nucleus, indicated successful uptake of FANAs by target Foxp3 expressing Treg cells.

### Example 4

### Evaluation of Foxp3 FANAs Effect on Foxp3 Protein Expression Level

The effect of Foxp3 FANA on Foxp3 protein expression level was evaluated via Western Blot to assess the ability of Foxp3 FANAs to inhibit Foxp3 expression at a protein level.

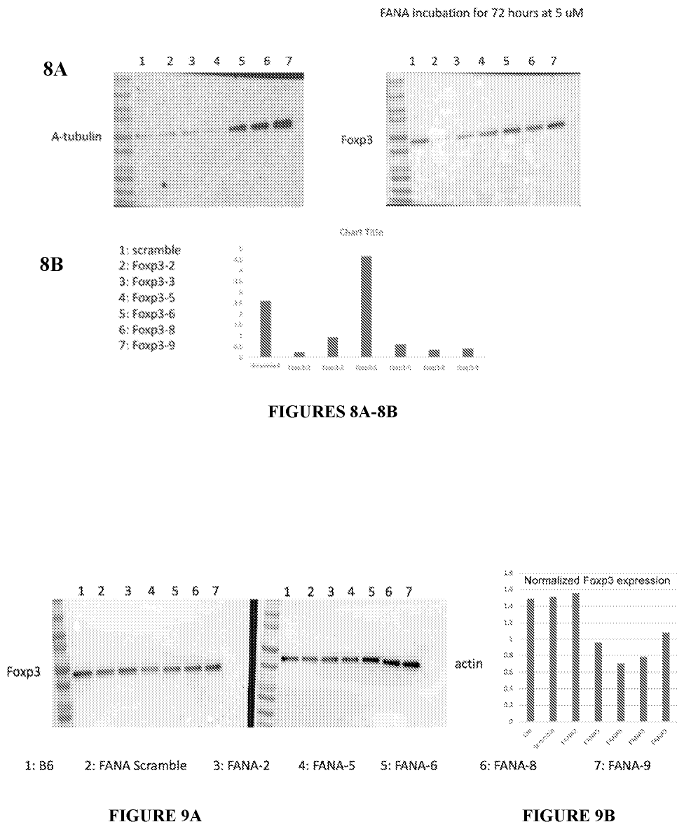

The in vitro effect of Foxp3 FANAs was evaluated after the cells were treated with 5 μM of several FANAs for 72 hours. As illustrated in FIGS. 8A-B, Foxp3 expression was measured along with a-tubulin expression as a loading control, which was used to normalize the Foxp3 expression. Most FANAs induced reduced expression of Foxp3 as compared to scrambled control.

The in vivo effect of Foxp3 FANAs was also evaluated. Mice were treated with three doses of 10 mg/kg of FANAs targeting Foxp3. 24 hours after the last injection the cells were harvested. As illustrated in FIGS. 9A-B, Foxp3 protein was measured and compared to actin as a control. FANA 5, 6, 8, and 9 reduced Foxp3 expression as compared to scrambled FANA and to the control.

### Example 5

### Evaluation of Foxp3 FANAs Effect on Treg Immune Suppressive Function

The immune suppressive function of Treg cells was assessed by flow cytometry, where the effect of Foxp3 FANA-treated Tregs on T effector cells was measured in a Treg immune suppression assay.

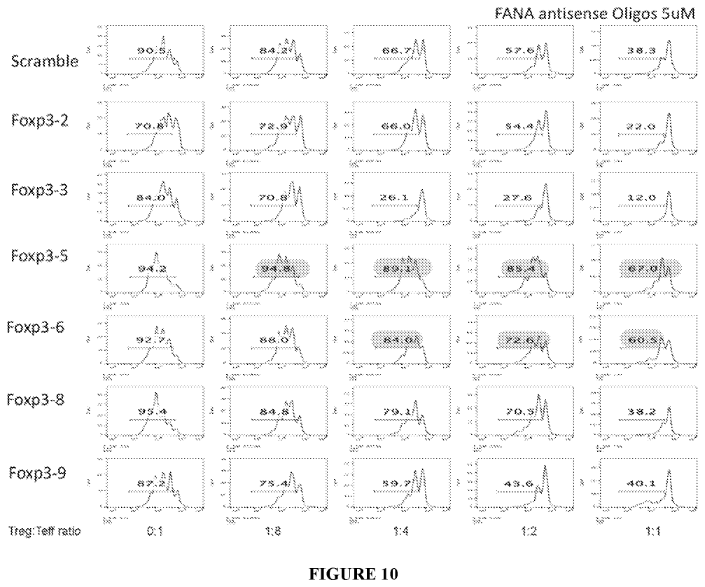

The in vitro effect of Foxp3 FANAs was evaluated after the treatment of Treg with 5 μm FANAs. Various ratios of Foxp3 FANAs treated Treg and T effector cells were then assessed, and the ability of Treg cells to immune suppress T effector was measured by evaluating the number of T effector cells. Efficient Foxp3 FANAs were identified by their ability to reduce Treg immune suppression of T effector cells. In FIG. 10, where each column indicated a ratio of Treg cells to T effector cells (TE, which are normal cytotoxic immune cells), when the percentage of Treg cells increased in the sample, the proliferation of TE cells was expected to decrease, because immune activation/proliferation was suppressed by Treg. As illustrated in the scramble control, without Treg involvement, T effector cells proliferated and constituted 90.5% of the population. As Treg cell number increased and the initial populations came closer to equal numbers of Tregs and TE cells (ratio 1:1), the proliferation of TE cells decreased to where only 38.3% constituted TE cells. Treatment of the cells with FANAs, and evaluation of those same percentages allowed FANAs capable of preventing Treg immune suppression to be identified. AUM-FANA-5 and AUM-FANA-6 both prevented immune suppression of T effector cells by Tregs, as can be seen by the highlighted percentages. In FIG. 10, even when increasing numbers of Tregs were added to the system, when Treg were treated with AUM-FANA-5 or AUM-FANA-6 TE function and activity were preserved, presumably because of the silencing Foxp3 in Treg cells and because of the blocking of their suppressive action.

### Example 6

### In Vivo Evaluation of the Effect of 50 mg/kg of Foxp3 FANA Oligonucleotides

The effects of a higher dose of 50 mg/kg of Foxp3 FANA oligonucleotides was evaluated in vivo by assessing several parameters such as the impact on the immune suppressive function of Treg cells, and the impact on the protein expression level of Foxp3.

Mice were injected intra-peritoneally once a day for 7 days with 50 mg/kg dose of AUM-FANA-5 or AUM-FANA-6, two of the most efficient Foxp3 FANAs as established per the previously presented data. 24 hrs after the last injections, their spleen and lymph nodes (LNs) were collected and Tregs were enumerated and evaluated.

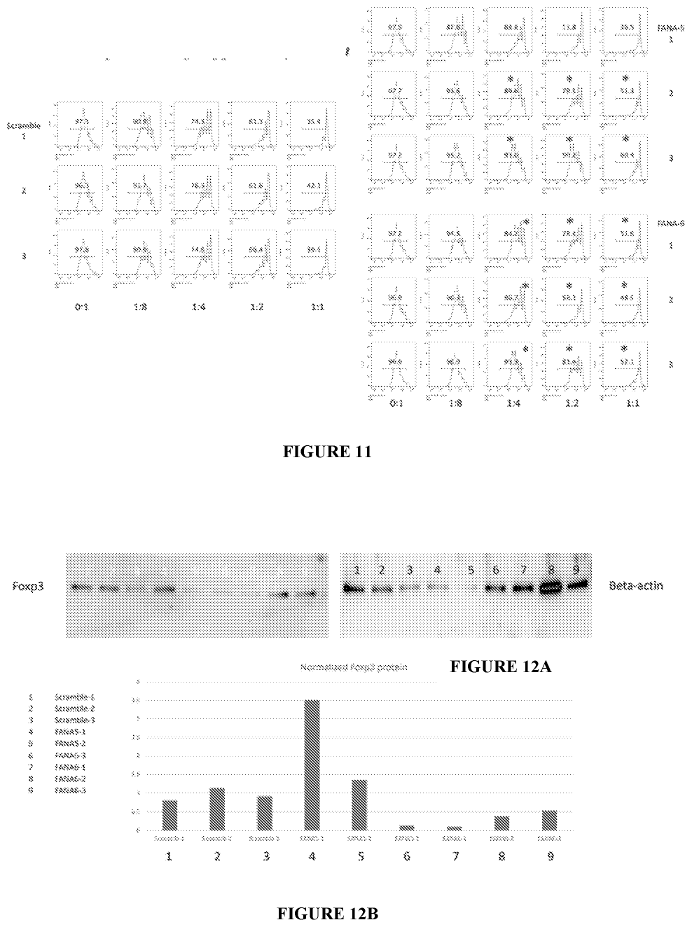

The ability of Treg to immune suppress T effector cells was measured by evaluating the number of T effector cells by flow cytometry. Tregs were subjected to a Treg suppression assay, where increasing number of Treg cells were incubated with normal cytotoxic immune cells, T effector (TE) cells. As the percentage of Treg cells increased in the sample, the proliferation of TE cells was expected to decrease because immune activation was suppressed by Treg. As illustrated in the control row in FIG. 11, without Treg involvement, TE cells proliferated and constituted 97.3% of the population. As Treg cells were increased and the initial populations came closer to equal numbers of Tregs and TE cells, the proliferation of TE cells was decreased to where only 35.4% constituted TE cells. Treatment of the cells with AUM-FANA-5 and AUM-FANA-6 were both found to prevent Treg mediated suppression of T effector cells (flow cytometry plots with stars), as measured percentages of T effector cells were higher than occurred in the control sample given the presence of specific ratios of Tregs. This dose was effective, where the 10 mg/kg dose three times was less effective. Daily doses of 50 mg/kg in vivo reduced Treg mediated immune suppression and restored immune system effector function.

Further, and as illustrated in FIGS. 12A-B, Foxp3 protein was measured and compared to beta-actin as a control. Western blot revealed that AUM-FANA-5 and AUM-FANA-6 both decreased Foxp3 protein expression in vivo as compared to scramble controls. Daily doses of 50 mg/kg FANAs in vivo reduced protein expression of Foxp3.

### Example 7

### In Vivo Evaluation of the Treatment of TC1 Mice with Foxp3 FANA Oligonucleotides

The effect of selected Foxp3 FANAs AUM-FANA-5 (SEQ ID NO:25) and AUM-FANA-6 (SEQ ID NO:26) on tumor growth was evaluated in vivo using the TC1 tumor model as described in Example 1. Further, the effects of AUM-FANA-5 and AUM-FANA-6 on intratumoral and intrasplenic Foxp3⁺ Treg were evaluated by flow cytometry.

TC1 cells were injected into mice on day 0, and tumors were allowed to grow until day 7. On day 7, groups of 10 mice each were randomly separated, and each mouse was treated daily with intra-peritoneal injection 50 mg/kg of scramble control, AUM-FANA-5, or AUM-FANA-6, for 14 days. Each day tumor size was measured and plotted.

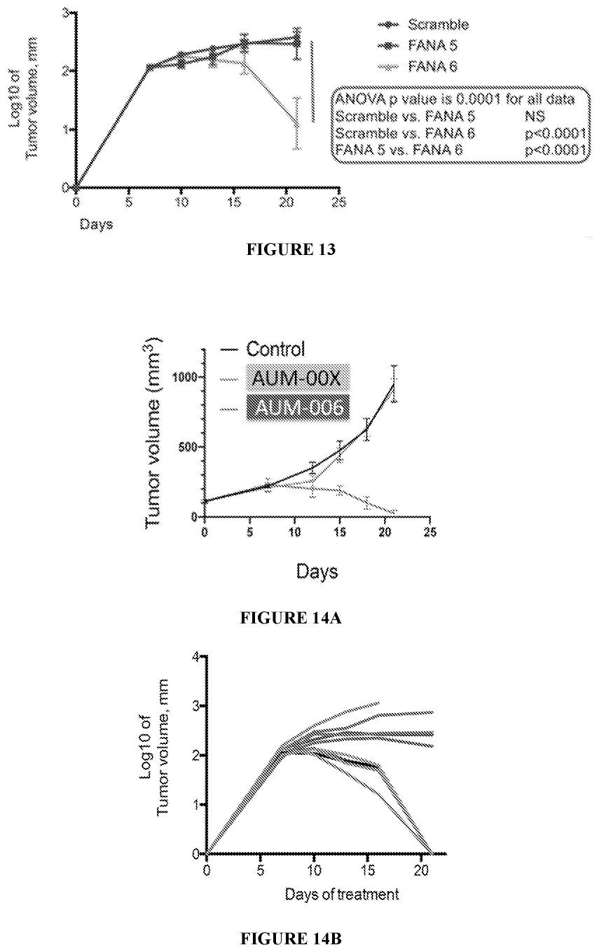

As illustrated in FIG. 13, on day 20, AUM-FANA-6 was found capable of significantly reducing tumor size as compared to both AUM-FANA-5 and the scramble control.

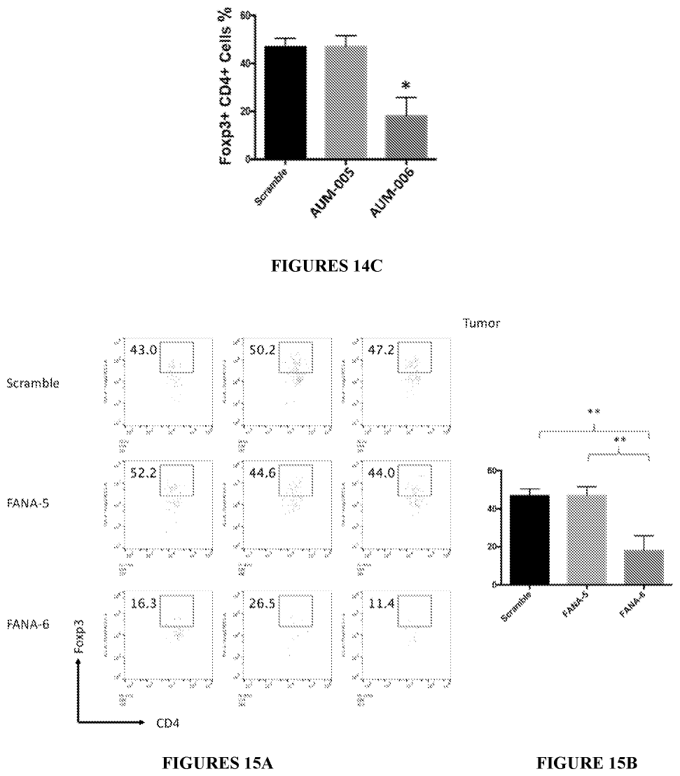

As further detailed in FIGS. 14A-C, it was found that Foxp3 ASO therapy impaired growth of TC1 lung tumors growing in syngeneic C57BL/6 mice. As illustrated in FIG. 14B, where each line represented a different mouse, it was found that tumors completely regressed and disappeared in 5 of the 10 AUM-FANA-6 treated mice, and slowed in several others. Daily doses of 50 mg/kg FANAs for two weeks considerably reduce tumor size in vivo, and AUM-FANA-6 was able to induce complete tumor regression in 5 out of 10 treated mice.

Further, the number of intratumoral Foxp3 expressing cells was measured by flow cytometry. After the final endpoint of the experiment, and after sacrifice of the animals, intratumoral cells were harvested and analyzed. As illustrated in FIGS. 15A-B, it was found that 50 mg/kg doses of FANAs, especially AUM-FANA-6 reduced the number of Foxp3 expressing cells in the tumors of treated mice, demonstrating that Foxp3 knockdown was successful, and contributed to the rejection of tumors in half of the AUM-FANA-6 treated mice.

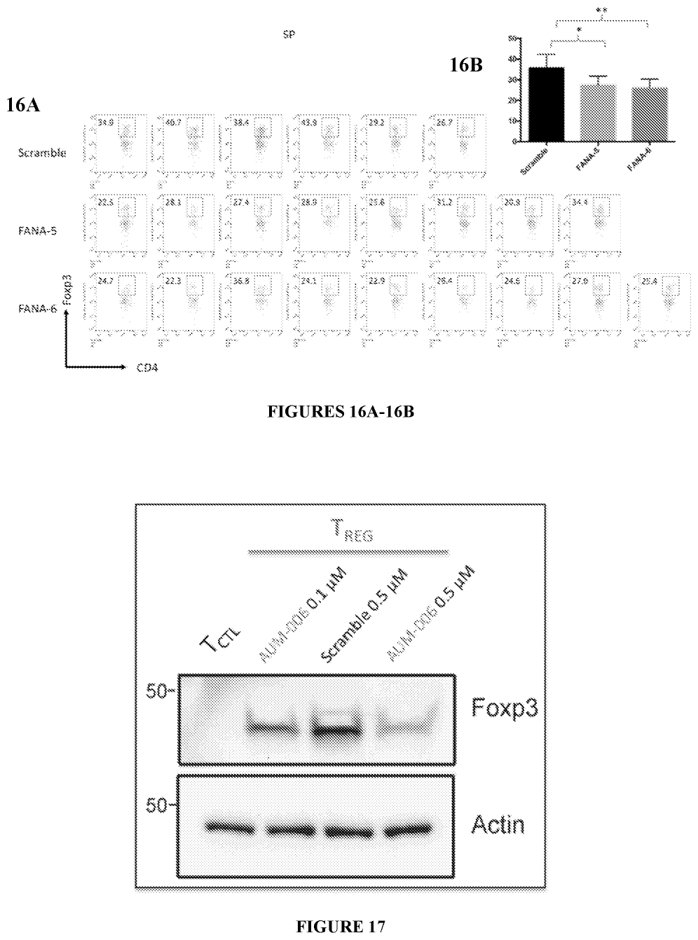

Additionally, the number of intrasplenic Foxp3 expressing cells was measured by flow cytometry. After the final endpoint of the experiment, and after sacrifice of the animals, intratumoral cells were harvested and analyzed. As illustrated in FIGS. 16A-B, it was found that 50 mg/kg doses of FANAs reduced the number of Foxp3 expressing cells in the spleens of treated mice, demonstrating that Foxp3 knockdown was successful.

### Example 8

### In Vivo Evaluation of the Treatment of TC1 Mice with Low Dose of Foxp3 FANA6-B Oligonucleotides

The effect selected Foxp3 FANAs, were evaluated in vitro and in vivo using lower doses of oligonucleotides.

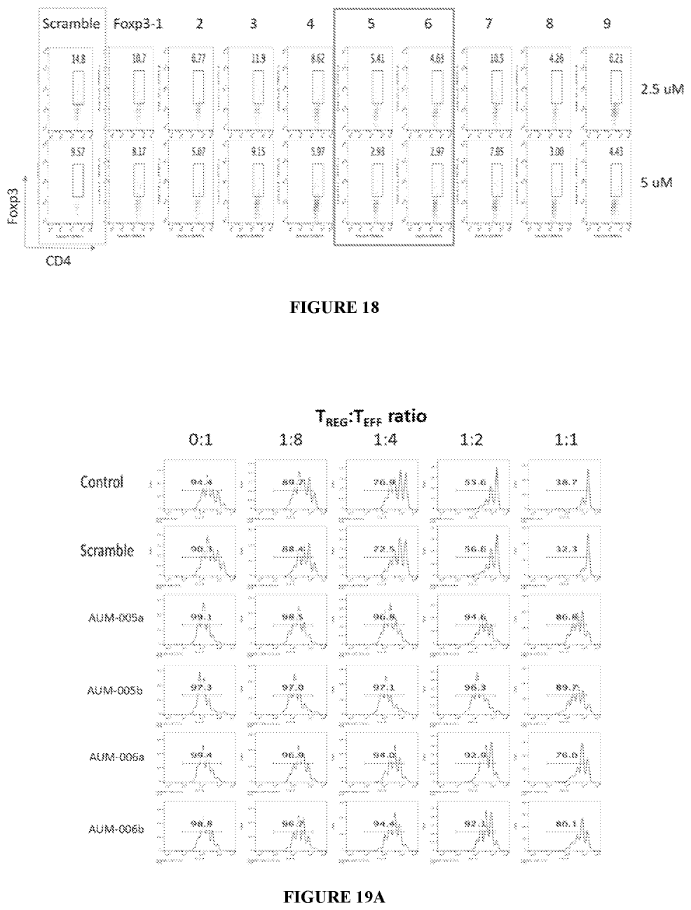

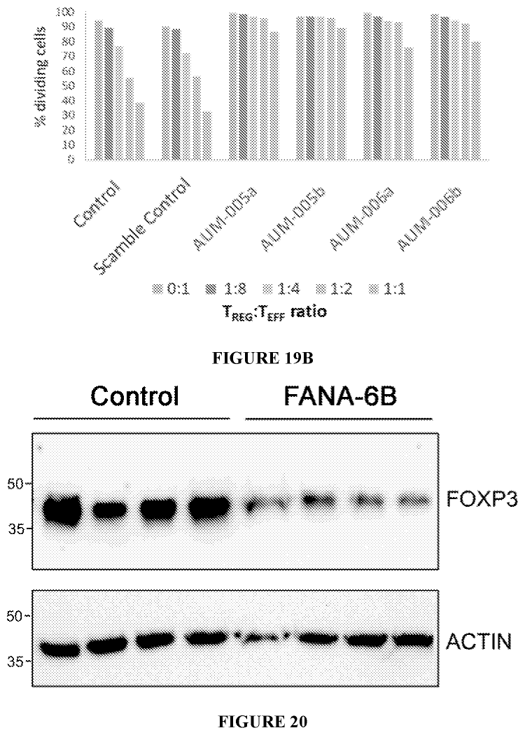

As illustrated in FIG. 17, it was demonstrated that AUM-FANA-6 (SEQ ID NO:26) was efficient to induce an in vitro Foxp3 knockdown. Further, the evaluation of the number of Foxp3+ splenic cells after treatment with various FANAs at 2.5 or 5 μM of oligonucleotides was evaluated by flow cytometry data on murine splenocytes treated with CD3 mAb. As illustrated in FIG. 18, anti-Foxp3 AUM-FANA-5 (SEQ ID NO:25) and AUM-FANA-6 (SEQ ID NO:26) reduced the population of Foxp3+ TREGS in murine splenocytes. Additionally, Treg suppressive function was evaluated in vitro after the treatment of the cells with AUM-FANA-5 or AUM-FANA-6. As shown in FIGS. 19A-B, AUM-FANA-5 or AUM-FANA-6 impaired murine TREG suppressive function, as illustrated by the sustained proliferation of conventional T cells induced by CD3 mAb when the cells are treated with the oligonucleotides, as compared to the non-treated cells (treatments had final FANA concentrations of 1 μM).

The in vivo analysis of lower doses of FANAs oligonucleotides was first evaluated in draining lymph nodes of tumor-bearing mice, using the TC1 tumor model, as described in Example 1. TC1 cells were injected into mice on day 0, and tumors were allowed to grow. Two groups of 4 mice each were randomly separated, and each mouse was treated daily with intra-peritoneal injection of scramble control, or AUM-FANA-6B (SEQ ID NO:304). On day 21, draining lymph nodes of tumor-bearing mice were collected and Foxp3 expression was evaluated by western blot. As illustrated in FIG. 20, AUM-FANA-6B was efficient at reducing Foxp3 expression in draining lymph nodes, as compared to the scramble control.

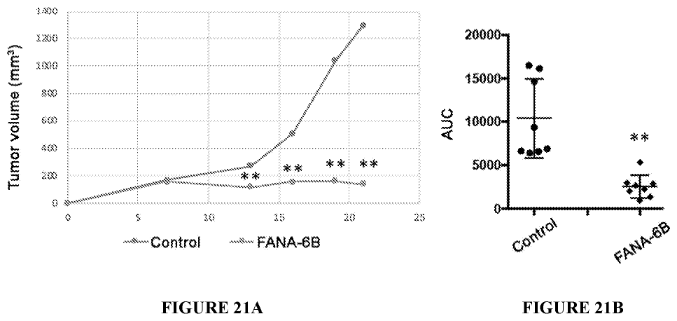

Further, the effects of FANA AUM-FANA-6B on tumor growth and anti-tumor immunity were evaluated in vivo using the TC1 tumor model as described in Example 1. TC1 cells were injected into mice on day 0, and tumors were allowed to grow until day 7. On day 7, groups of 8 mice each were randomly separated, and each mouse was treated daily with intra-peritoneal injection 25 mg/kg of scramble control, or AUM-FANA-6B, for 14 days. Each day tumor size was measured and plotted. As illustrated in FIGS. 21A-B, on day 21, 25 mg/kg/day of AUM-FANA-6B was found capable of significantly reducing tumor size as compared to the scramble control, showing that Foxp3 AUM-FANA-6B impaired growth of lung tumors.

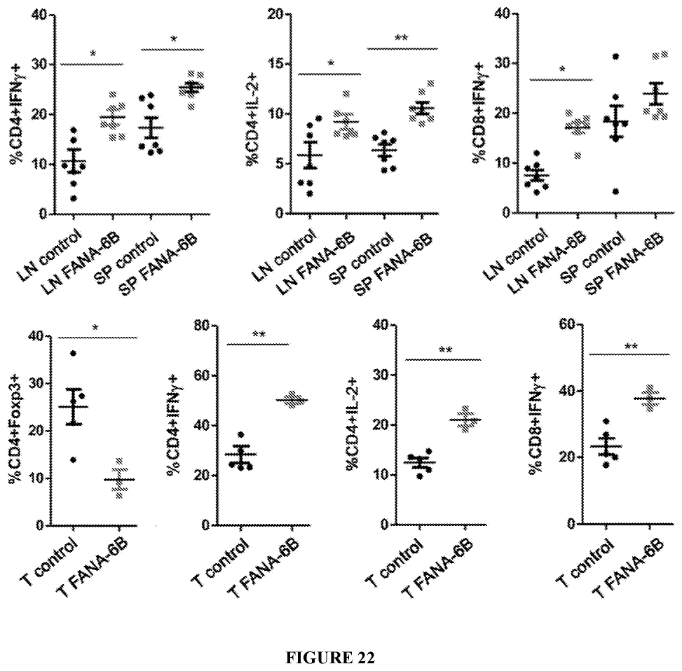

The percentages of CD4⁺IFN-g⁺, CD4⁺IL-2^{+,} CD8³⁰ IFN-g⁺, and Foxp3⁺ Treg cells were evaluated in lymphoid tissues and at tumor sites. As illustrated in FIG. 22, 25 mg/kg/day of AUM-FANA-6B was found capable of significantly increasing CD4⁺IFN-g⁺, CD4⁺IL-2⁺, and CD8⁺IFN-g⁺ T cells in both lymphoid tissues and tumor sited; and was also found capable of decreasing intra-tumoral Foxp3⁺ Treg cells; showing that Foxp3 FANA-6B promoted anti-tumor immunity.

Although the invention has been described with reference to the above examples, it will be understood that modifications and variations are encompassed within the spirit and scope of the invention. Accordingly, the invention is limited only by the following claims.

1-38. (canceled)

39. A modified antisense oligonucleotide (AON) comprising at least one 2′-deoxy-2′-fluoro-β-D-arabinonucleotide (2′-FANA modified nucleotide), wherein the AON binds to a Foxp3 mRNA.

40. The AON of claim 39, which is a hybrid chimera AON comprising at least one 2′-FANA modified nucleotide and at least one unmodified deoxyribonucleotide, wherein the AON is a 2′-FANA AON.

41. The AON of claim 40, wherein the 2′-FANA modified nucleotides are positioned according to any of Formulas 1-16.

42. The AON of claim 40, wherein internucleotide linkages between nucleotides of the 2′-FANA modified nucleotides are selected from the group consisting of phosphodiester bonds, phosphotriester bonds, phosphorothioate bonds (5′O—P(S)O—3O—, 5′S—P(O)(—3′—O—, and 5′O—P(O)O—3′S—), phosphorodithioate bonds, Rp-phosphorothioate bonds, Sp-phosphorothioate bonds, boranophosphate bonds, methylene bonds(methylimino), amide bonds (3′—CH₂—CO—NH—5′ and 3′—CH₂—NH—CO—5′), methylphosphonate bonds, 3′-thioformacetal bonds, (3′S—CH₂—O5′), amide bonds (3′CH₂—C(O)NH—5′), phosphoramidate groups, and a combination thereof.

43. The AON of claim 40, wherein the 2′-FANA AON comprises from about 0 to about 20 2′-deoxy-2′-fluoro-β-D-arabinonucleotide at the 5′-end and from about 0 to about 20 2′-deoxy-2′-fluoro-β-D-arabinonucleotide at the 3′-end, flanking a sequence comprising from about 0 to about 20 deoxyribonucleotide residues.

44. The AON of claim 41, wherein the 2′-FANA AON comprises at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID NOs: 193-302, or a sequence complimentary thereto.

45. A pharmaceutical composition comprising a modified antisense oligonucleotide (AON) comprising at least one 2′-deoxy-2′-fluoro-β-D-arabinonucleotide (2′-FANA modified nucleotide) and a pharmaceutically acceptable carrier, wherein the AON binds to a Foxp3 mRNA.

46. The composition of claim 45, wherein the AON is a hybrid chimera AON comprising at least one 2′-FANA modified nucleotide and at least one unmodified deoxyribonucleotide, and wherein the AON is a 2′-FANA AON.

47. The composition of claim 46, wherein the 2′-FANA AON comprises from about 0 to about 20 2′-deoxy-2′-fluoro-β-D-arabinonucleotide at the 5′-end and from about 0 to about 20 2′-deoxy-2′-fluoro-β-D-arabinonucleotide at the 3′-end, flanking a sequence comprising from about 0 to about 20 deoxyribonucleotide residues.

48. The composition of claim 47, wherein the 2′-FANA AON comprises at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID NOs: 193-302, or a sequence complimentary thereto.

49. A method of treating cancer in a subject in need thereof comprising administering to the subject at least one antisense oligonucleotide (AON), wherein the AON binds to a Foxp3 mRNA, and wherein the AON comprises at least one 2′-deoxy-2′-fluoro-β-D-arabinonucleotide (2′-FANA modified nucleotide), thereby treating the cancer.

50. The method of claim 49, wherein the AON reduces expression level of a Foxp3 gene.

51. The method of claim 49, wherein the AON is a hybrid chimera AON comprising at least one 2′-FANA modified nucleotide and at least one unmodified deoxyribonucleotide, and wherein the AON is a 2′-FANA AON.

52. The method of claim 51, wherein the 2′-FANA AON comprises at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NOs: 1-9, SEQ ID NOs: 11-19, SEQ ID NOs: 21-29, SEQ ID NOs: 31-138, SEQ ID NOs: 139-192, SEQ ID NOs: 193-302, or a sequence complimentary thereto.

53. The method of claim 51, wherein the 2′-FANA AON increases anti-tumor immunity in the subject.

54. The method of claim 51, wherein the 2′-FANA AON decreases the activity of a regulatory T cell (Treg) and/or increases the activity of an immune cell.

55. The method of claim 49, further comprising administering an immunotherapeutic agent, a chemotherapeutic agent and/or a radiotherapy.

56. The method of claim 55, wherein the immunotherapeutic agent and/or chemotherapeutic agent is selected from the group consisting of a checkpoint inhibitor, vaccine, chimeric antigen receptor (CAR)-T cell therapy, anti-PD-1 antibody (Nivolumab or Pembrolizumab), anti-PD-L1 antibody (Atezolizumab, Avelumab or Durvalumab) and a combination thereof.

57. The method of claim 55, wherein the immunotherapeutic agent, chemotherapeutic agent and/or radiotherapy is administered prior to, simultaneously with, or after the administration of the AON.

58. The method of claim 49, wherein the cancer is selected from the group consisting of breast, liver, ovarian, pancreatic, lung cancer, melanoma and glioblastoma.
